(19) United States
(12) Patent Application Publication
(10) Pub. No.: US 2012/0089220 A1
(43) Pub. Date: Apr. 12, 2012

(54) MICROCATHETER

(75) Inventor: Alessandro Lualdi, Milan (IT)

(73) Assignee: E.V.R. Endovascular Researches S.A., Luxembourg (LU)

(21) Appl. No.: 13/226,428

(22) Filed: Sep. 6, 2011

Related U.S. Application Data
(63) Continuation-in-part of application No. 10/512,245, filed on May 12, 2005, now abandoned.
Application No. 10/512,245 is a continuation-in-part of application No. 12/471,281, filed on May 22, 2009, now Pat. No. 8,012,199.
Application No. 12/471,281 is a division of application No. 10/204,251, filed on Nov. 26, 2002, now Pat. No. 7,540,879.

(30) Foreign Application Priority Data
Feb. 18, 2000 (IT) 00200572.6
Apr. 22, 2002 (IT) MI2002A000860

(51) Int. Cl. A61F 2/82

(52) U.S. Cl. 623/1.35

(57) ABSTRACT

An endolumenal for delivering and positioning an endolumenal expandable prosthesis for a bifurcation is provided. The endolumenal device includes a guidewire tracking device and an elongated body that has a central longitudinal axis and an expansion device configured to expand symmetrically relative to the central longitudinal axis. The guidewire tracking device comprises a single guidewire lumen disposed within a wall structure of the expansion device. The guidewire tracking device has at least three distal ports extending through the wall structure of the expansion device. The guidewire lumen includes a distal apical port in an approximately central position relative to the expansion device, considered in cross section at right angles to the central longitudinal axis.

### CROSS-REFERENCE TO RELATED APPLICATIONS

This application is a continuation-in-part of U.S. application Ser. No. 10/512,245, filed May 12, 2005, which is a U.S. National Phase of International Application No.: PCT/IB03/01178, filed Mar. 27, 2003 designating the U.S. and published in English on Oct. 30, 2003 as WO 03/088871, which claims the benefit of Italian Patent Application No. MI2002A000860, filed Apr. 22, 2002, and is a continuation-in-part of U.S. application Ser. No. 12/471,281, filed May 22, 2009, which is a divisional of U.S. application Ser. No. 10/204,251, filed Nov. 26, 2002, which is a U.S. National Phase of International Application No. PCT/EP00/13964, filed Dec. 19, 2000 designating the U.S. and published in English on Aug. 23, 2001 as WO 01/60284, which claims the benefit of Italian Patent Application No. 00200572.6, filed Feb. 18, 2000. Each of the applications listed above is incorporated by reference herein in its entirely.

### BACKGROUND OF THE INVENTIONS

1. Field of the Invention

This invention is directed to endolumenal devices for delivering and deploying endolumenal expandable prostheses.

2. Description of the Related Art

Endolumenal devices for delivering and deploying an endolumenal expandable prosthesis are used for delivering and deploying prostheses or stents endolumenally within conduit systems, for example vessels carrying body fluids and, in particular, lumens in the bodies of human beings and animals. Such vessels for the transportation of fluids are, for example, arterial blood vessels, such as coronary, mesenteric, peripheral and cerebral arteries, veins or gastrointestinal tracts.

Using the abovementioned devices it is possible to implant endolumenal prostheses, or stents, in a vessel in which arteriosclerotic plaque, or arteriostenosis, has at least partially occluded the lumen. The prosthesis forms a radial support for the surrounding wall of the lumen and prevents it partially or completely occluding again, once it has been dilated by a balloon or other expansion means. These operations are carried out using known angioplasty techniques. Techniques of this type are, for example, described in the publication “The New Manual of Interventional Cardiology” edited by Mark Freed, Cindy Grines and Robert D. Safian, Division of Cardiology at William Beaumont Hospital, Royal Oak, Mich.; Physicians' Press 1996.

The widespread use of these techniques is considerably limited by the significant difficulties presented by the known endolumenal devices when they are used on vascular bifurcations of the system of conduits (bifurcation lesions).

It is known that operations on bifurcation lesions are frequently subject to procedural failures and acute complications, because the known devices cause occlusion of that branch of the bifurcation which originates near the area in which the prosthesis is fitted.

In particular, the activation of a balloon in a first branch of the bifurcation can cause the atheromatous material of the plaques to be displaced until it obstructs the ostium of a second branch of the bifurcation, (a problem known as snow-plow or plaque-shifting).

Due to the abovementioned snow-plough or plaque-shifting, the ostium of the occluded branch must again be rendered accessible, or regained, such as by re-introducing a guidewire through a barrier consisting of the displaced plaque.

In some cases, it is necessary, following the implanting of the first prosthesis, to insert a second guidewire and a second prosthesis into the occluded branch, passing through the meshes or struts of the first prosthesis. Even when it is possible to regain access to the occluded branch, the operation becomes extremely lengthy and, in any case, the results depend very much on the experience of the surgeon. As a result, where the above-described bifurcation lesions are present, the operation must be carried out in highly qualified centers, fully equipped for cardiac surgery, that may be called upon urgently in the case of damage following occlusions caused during the endolumenal operation and lack of success in regaining the ostium.

Due to the abovementioned difficulties, the use of stents with wide apertures to allow the passage of the prosthesis and the introduction of a guidewire into the branches has been proposed. However, these wide apertures can give rise to an increase in prolapse of plaque through the meshes and, therefore, imperfect vascularization and increased probability of re-stenosis.

One alternative that has been proposed is the simultaneous use of two devices fitted with expansion means for the simultaneous insertion of two stents in each of the branches of the bifurcation (paired or kissing devices), or of a single bifurcated stent.

This known solution however is very bulky and difficult to maneuver and can only be used in large vessels and in neighboring portions. In other words, it is impossible to use this known solution in peripheral branches, where the formation of atheromes or arteriosclerotic plaques is more likely. Furthermore, in order to insert the known paired devices it is necessary to use large-diameter guide catheters. The greater bulk of the paired devices occludes the vessel during insertion causing ischemia during the procedure and making it impossible to inject a contrast medium which is useful for visualizing the path for the correct positioning, first of the guidewire and then of the endolumenal devices fitted with the prosthesis.

The use of paired devices also lacks versatility, above all in the case of a single bifurcated stent, since the three vascular segments which make up the bifurcation—the proximal principal vessel, the principal vessel distal to the bifurcation and the secondary vessel, or side branch—may be of very different bores with lesions of varying lengths. It is therefore impossible at present to prepare a range of bifurcated stents which can be adapted to all the possible anatomical and pathological variables. It must also be noted that these bifurcated stents, of fixed dimensions, often occlude other branches near the bifurcation lesions, with consequent ischemia or incomplete revascularization.

It is therefore evident that not all bifurcation lesions, and in particular coronary bifurcation lesions, can be dealt with percutaneously.

### SUMMARY OF THE INVENTIONS

The above considerations show that the need for endolumenal devices for delivering and deploying an endolumenal expandable prosthesis, which can reach both the branches of a bifurcation safely and rapidly, is widely felt. A need is likewise felt to be able to fit endolumenal prostheses which are morphologically adaptable to the anatomy and to the pathology of the proximal and distal portions of the branches of the bifurcation. In other words, it is desirable to be able to deal with all types of lesions using a single endolumenal device, of the type described above, capable of adapting to a vast range of vessel diameters and lesions of any length. The endolumenal devices must also ensure the accurate deployment of various prostheses, to provide ample coverage of the bifurcation in order to prevent protrusion of plaque between the various prostheses fitted and to prevent the formation of re-stenosis.

Therefore, an object of this invention is to devise and make available an endolumenal device of the type specified above, which will meet the needs described above and, at the same time, make it possible to avoid all the pitfalls outlined.

These objects are achieved in one embodiment by an endolumenal device for delivering and positioning an endolumenal expandable prosthesis for a bifurcation between a principal conduit and at least one secondary conduit. The endolumenal device includes an elongated body and a guidewire lumen. The elongate body has a central longitudinal axis, a proximal portion, and a distal portion. The distal portion of the elongated body has an expansion device configured to expand symmetrically relative to the central longitudinal axis and a longitudinally extended active portion removably engageable with the endolumenal expandable prosthesis. The longitudinally extended active portion is adapted to adjust the prosthesis from a radially collapsed condition to a radially expanded condition. The guidewire lumen extends at least partially along the elongated body. The guidewire lumen preferably comprises a single guidewire lumen disposed within a wall structure of the expansion device. The guidewire tracking device has a plurality of, e.g., preferably three or more distal ports extending through the wall structure of the expansion device and adapted to receive therethrough a portion of at least one guidewire positionable with the distal portion in the principal conduit or in the at least one secondary conduit. The guidewire lumen includes a distal apical port in an approximately central position relative to the expansion device, considered in cross section at right angles to the central longitudinal axis. In some embodiments, a central region of the expansion device comprises a free volume. For example, the central region can be devoid of a pipe or elongate body having a lumen or other structure or any other member disposed therein for adding rigidity or pushability.

The combinations of features set forth above solve a number of significant problems. For example, this combination of features can produce an endolumenal device that is able to treat very small vessels. Such vessels can be very difficult to treat since they are very small and can only be reached through tortuous paths. Also, when delivering an endolumenal device to a very small vessel through tortuous passages, an endolumenal device will often be in a random and non-optimal orientation. Therefore, to be able to rotate the device when delivered is a problem. Orienting an endolumenal device through such a passage is made more difficult by the tortuousity of the vessel segment. Moreover, many catheters are single purpose and thus greatly enlarge inventory requirements for end users. This a device that would be able to be multipurpose would be extremely advantageous. Moreover, many catheter devices are very complicated, which leads to greater cost. It would be desirable to eliminate excess components if possible. Other embodiments set forth herein below also solve these many challenges and problems for small vessel bifurcation access with catheters and other endolumenal devices.

In another embodiment, a catheter assembly can be provided that has an elongate body and an expansion device coupled with a distal portion of the elongate body. The expansion device comprises a wall surrounding a cavity. The catheter assembly has a single guidewire lumen that extends from a proximal port to a distal port and that also extends at least partially within the wall of the expansion device. The single guidewire lumen has a plurality of side ports disposed along the expansion device that are sized to provide access for a guidewire to the guidewire lumen. The expansion device is expandable such that the wall is substantially symmetrically disposed around a longitudinal axis extending through the distal port of the guidewire lumen and such that the side ports move from a first radial position to a second radial position, the second radial position being radially farther away from the longitudinal axis than the first radial position.

In another embodiment, a method is provided for treating a patient, comprising. For example, a catheter assembly can be provided that has an elongate body and an expansion device coupled with a distal portion of the elongate body. The expansion device comprises a wall surrounding a cavity. The catheter assembly has a single guidewire lumen that extends from a proximal port to a distal port and that also extends at least partially within the wall of the expansion device. The single guidewire lumen has a plurality of side ports disposed along the expansion device that are sized to provide access for a guidewire to the guidewire lumen. A guidewire is positioned in the vasculature of a patient such that a distal portion thereof is disposed in a portion of a first blood vessel located adjacent to and distal of a bifurcation. The catheter assembly is positioned over the guidewire such that the distal portion of the guidewire extends through one of the side-ports and into the portion of the first blood vessel while at least a portion of the expansion device is located in a portion of a second blood vessel located adjacent to and distal of the bifurcation. The expansion device is expanded such that the wall is substantially symmetrically disposed around a longitudinal axis extending through the distal port of the guidewire lumen and such that the side ports move radially away from the longitudinal axis.

In another embodiment, a method of manufacturing an endolumenal device is provided. For example, an elongate member can be provided that has a substantially continuous construction including a first lumen and a second lumen. At least one of the first lumen and second lumens of the elongate members can be formed into functional catheter lumens by applying pressure to at least one of the first or second lumens under controlled temperature conditions.

### BRIEF DESCRIPTION OF THE DRAWINGS

Further characteristics and advantages of endolumenal devices according to this application will become evident from the description that follows of some preferred embodiments, which are given purely by way of example and without implying any limitation, with reference to the enclosed drawings, in which:

FIG. 1 is a plan view of an endolumenal device according to some embodiments;

FIG. 2 is a cross-sectional view of a distal portion of an endolumenal device similar to FIG. 1;

FIG. 2A is a side view of the distal portion of FIG. 2 shown in an unexpanded configuration, which can be configured for low profile delivery;

FIG. 2B is a cross-section view of the unexpanded distal portion of FIGS. 1-2A taken through section plane 2B-2B;

FIG. 3 is a detail view of a variation of a coupling region for the endolumenal device of FIG. 1, illustrating various techniques for connecting a proximal portion with a distal portion of an endolumenal device;

FIG. 4 is a detail view of a tip region of the endolumenal device of FIG. 1 illustrating various guidewire port locations;

FIG. 5 is a perspective view of a proximal portion of an expansion device illustrating one internal lumenal arrangement;

FIG. 6 is a perspective view of a distal portion of an expansion device of an endolumenal device, illustrating a tip portion and apical port;

FIGS. 7-10 illustrate various techniques for forming an expansion device for the endolumenal device described herein;

FIG. 11 illustrates another embodiment of an endolumenal device including first and second guidewire lumens with first and second rapid exchange ports;

FIG. 12 illustrates an endolumenal device and guidewire arrangement that is useful in performing treatment of a lesion at an ostial bifurcation;

FIG. 13 illustrates an endolumenal device and guidewire arrangement that is useful in performing treatment of a lesion at a Y-bifurcation;

FIG. 14 illustrates an endolumenal device and two guidewire arrangement that is useful in performing treatment of a lesion at an T-bifurcation;

FIG. 14A illustrates one method for advancing an endolumenal device to provide for placement of a stent of a relatively long length distal of a branch, where two guides are positioned in different portions of a guidewire lumen and where the wires enter or exit the lumen at the same port;

FIG. 14B illustrates one method for advancing an endolumenal device to provide for placement of a stent of a relatively short length distal of a branch, where two guides are positioned in different portions of a guidewire lumen and where the wires enter or exit the lumen at the same port;

FIG. 14C illustrates one method for advancing an endolumenal device to position the device generally proximal of a branch, where two guides are positioned in different portions of a guidewire lumen and where the wires enter or exit the lumen at the same port;

FIG. 14D illustrates one method for advancing an endolumenal device to provide for placement of a stent of a relatively long length distal of a branch, where two guides are positioned in different portions of a guidewire lumen and where the wires enter or exit the lumen at different ports;

FIG. 14E illustrates one method for advancing an endolumenal device to provide for placement of a stent of a relatively short length distal of a branch, where two guides are positioned in different portions of a guidewire lumen and where the wires enter or exit the lumen at different ports;

FIG. 14F illustrates one method for advancing an endolumenal device to position the device generally proximal of a branch, where two guides are positioned in different portions of a guidewire lumen and where the wires enter or exit the lumen at different ports;

FIG. 15 is a cross-sectional view of another embodiment of an endolumenal device where an expansion device and elongate neck portion comprise a monolithic construction;

FIG. 16 shows a partially sectioned view of the endolumenal device fitted with a prosthesis;

FIGS. 17 and 18 show a view from beneath, and a side view, of a detail of the device of FIG. 16;

FIGS. 19 and 19a show the enlarged cross section on IV-IV of the device of FIG. 17, according to two embodiments;

FIG. 20 shows an end view along the arrow V of the endolumenal device of FIG. 18;

FIGS. 21a and 21b show a partially sectioned view of the device of FIG. 16 during two stages of use;

FIGS. 22 and 23 show a view from beneath, and a side view, of a detail of an endolumenal device according to a second embodiment;

FIG. 24 shows the enlarged cross section on IX-IX of the device of FIG. 22;

FIG. 25 shows a front view along the arrow X of the device of FIG. 23;

FIGS. 26a and 26b show a partially sectioned perspective view of the device of FIG. 22 during two stages of use;

FIGS. 27 to 32c show a section through a ‘T bifurcation’ during eight stages in the deploying of endolumenal prostheses;

FIGS. 32d and 32e show in section two alternative stages in the deploying of prostheses in the bifurcation shown in FIG. 32c;

FIGS. 33 to 38 show a cross portion through a ‘Y bifurcation’ during six stages in the deploying of endolumenal prostheses;

FIGS. 39 and 40 show a view from beneath, and a side view, of an endolumenal device provided with two distal ports at the distal end of the body beyond the prosthesis;

FIG. 41 shows an enlarged section on XXVI [symbol] XXVI through the device of FIG. 39;

FIG. 42 shows a view along the arrow XXVII of the device of FIG. 40;

FIGS. 43 and 44 show a view from beneath, and a side view partially sectioned, of details of an endolumenal device having a single guidewire lumen associated to distal ports at the distal end of the body beyond the prosthesis;

FIG. 45 shows a view along the arrow XXX of the device of FIG. 44;

FIGS. 46 and 47 show a view from beneath, and a side view partially sectioned, of details of an endolumenal device having a plurality of guidewire lumens associated to a plurality of distal ports;

FIGS. 48 and 49 show a perspective view and a side view, in partial section, of an endolumenal device having a single guidewire lumen associated to an apical distal port and a plurality of distal ports spaced out along the body;

FIGS. 50 and 51 show a view from beneath, and a side view partially sectioned, of details of an endolumenal device having a first guidewire lumen associated to an apical distal port and a second guidewire lumen associated to a plurality of distal ports spaced out along the body;

FIGS. 52, 53 and 54 show a view from beneath, and a side view partially sectioned, and an enlarged sectioned prospective of details of an endolumenal device having a fissure suitable for realizing a distal port;

FIG. 55 shows a perspective view, partially sectioned, during a stage in the slipping of a guidewire proximal end into the body fissure of the device of FIG. 53;

FIGS. 56 to 60 show a cross portion through a vessel during five stages in the deploying of an embolization containment ‘device and of an endolumenal prosthesis;

FIG. 57c shows a detail in an enlarged scale of FIG. 57a;

FIG. 57b shows a cross portion through a vessel during a stages in the deploying of an embolization containment device according to a further embodiment;

FIGS. 61 to 66 show a cross portion through a bifurcation during six stages in the deploying of embolization containment devices and of an endolumenal prosthesis;

FIG. 67 shows a cross portion through the coronary ostium during a stage in the deploying of an endolumenal prosthesis;

FIGS. 68 to 70 show a cross portion through a bifurcation during three stages in the deploying of endolumenal prostheses by means of two endolumenal devices reciprocally connected through a guidewire;

FIG. 71 shows a perspective view, partially sectioned, of an endolumenal device wearing a stent provided with a differentiated spatial behavior.

FIG. 72 is a perspective view of a portion of a possible embodiment of an endolumenal device sectioned at two points along its longitudinal length;

FIG. 73 is a longitudinal section through a possible embodiment of the device shown in FIG. 72;

FIG. 74 is a perspective view of the device shown in FIG. 32, sectioned on XXXIII-XXXIII as shown in FIG. 73;

FIG. 75 is a longitudinal section through a possible further embodiment of the device shown in FIG. 72;

FIG. 76 is a perspective view of the device shown in FIG. 75, sectioned on XXXV-XXXV as shown in FIG. 75;

FIG. 77 shows an enlarged cross section through the device shown in FIG. 73 or 75 on XXXVI-XXXVI as shown in FIG. 73 or 75;

FIG. 78 shows the enlarged cross section of FIG. 77 illustrating the device into which a guidewire has been inserted; and

FIG. 79 is a cross section through an endolumenal device in another possible embodiment.

### DETAILED DESCRIPTION OF THE PREFERRED EMBODIMENT

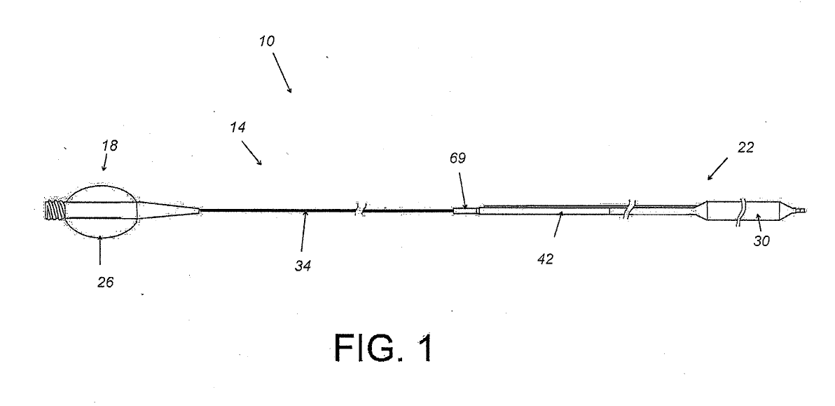

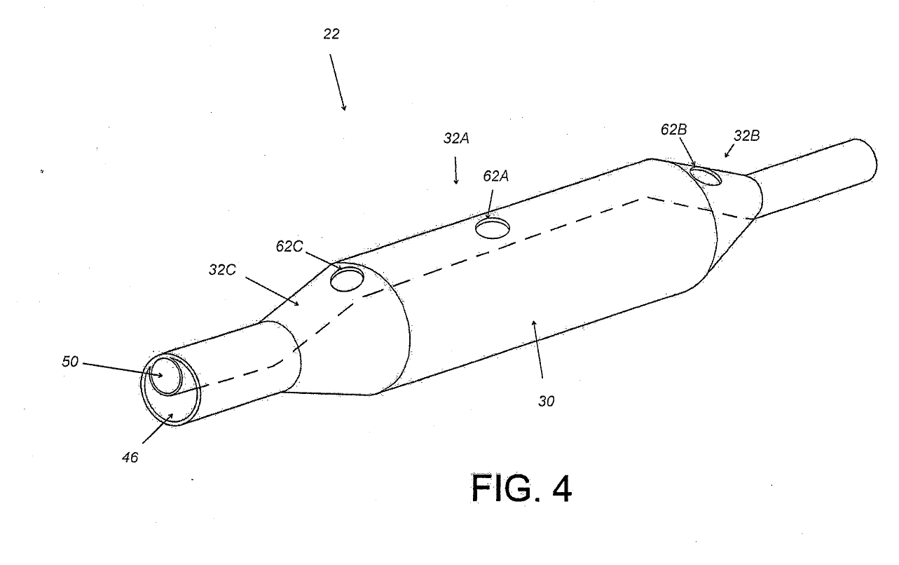

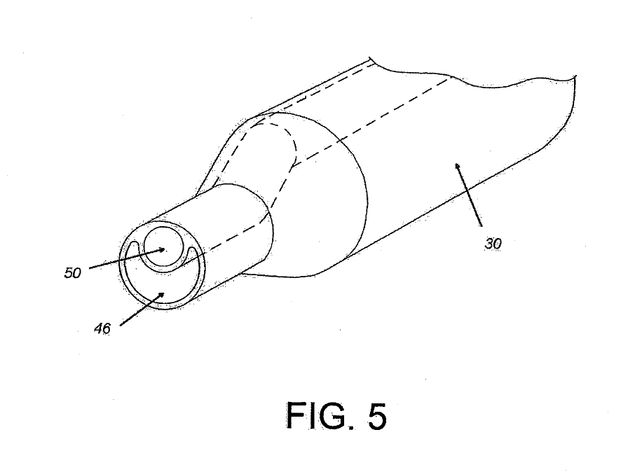

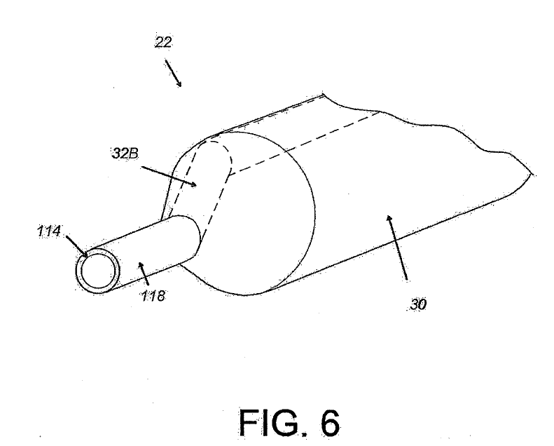

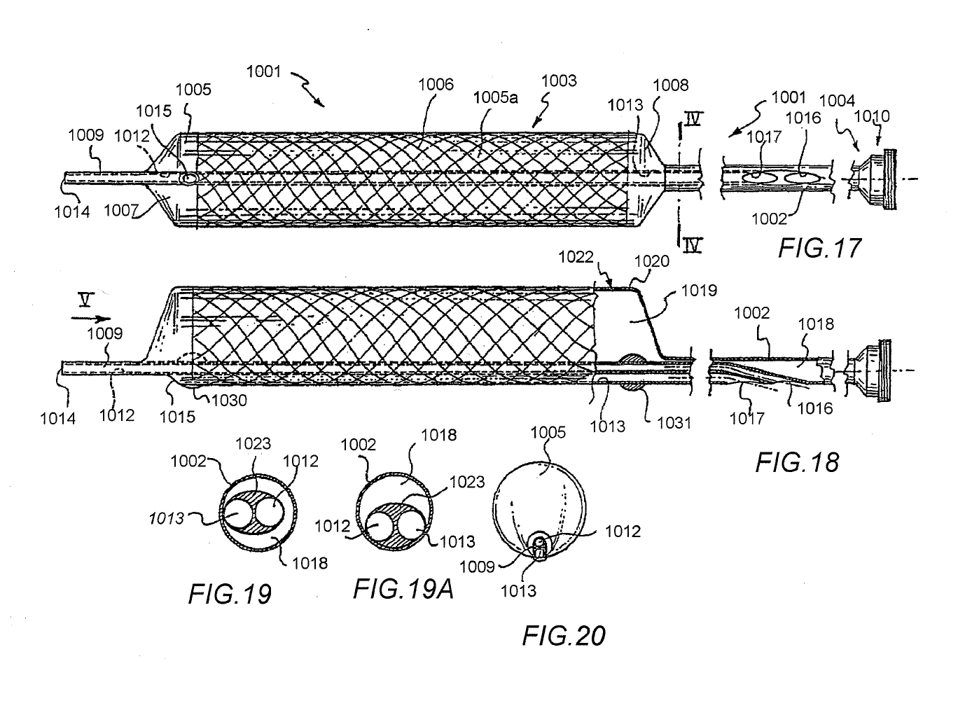

This application describes various embodiments of endovascular devices that can be used to access vessels at or adjacent to vascular bifurcations. Such devices that are particularly useful for smaller vessels are described in connection with FIGS. 1-16. Devices that are particularly useful for larger vessels are described in connection with FIGS. 17-79. Where the below disclosure is related to methods of use or manufacture, many concepts are applicable to all of the various endolumenal devices and such disclosure can be freely combined to provide for additional systems, kits, and methods, and modifications.

### I. Catheter Apparatuses and Methods for Accessing Smaller Bifurcation and Vessels

FIG. 1 shows an endolumenal device 10, commonly referred to as a catheter or a catheter assembly, that is useful in treating lesions at a vascular bifurcation. As will be discussed in greater detail below, the catheter assembly 10 is useful in very small blood vessels that cannot be treated percutaneously using conventional balloon catheters. As is discussed in greater detail below, the embodiments described herein are made more compact and lower profile by integrating and merging multiple constructs together to provide a lower profile construct. In some cases, a monolithic construct is provided between components that have previously been separate components.

The catheter assembly 10 includes an elongated body 14 extending between a proximal portion 18 and a distal portion 22. The proximal portion 18 of the elongate body 14 can be fitted with a connector 26 for coupling with an external device, such as a source of inflation media. The connector 26 can be any suitable connector, such as a luer connector or fitting.

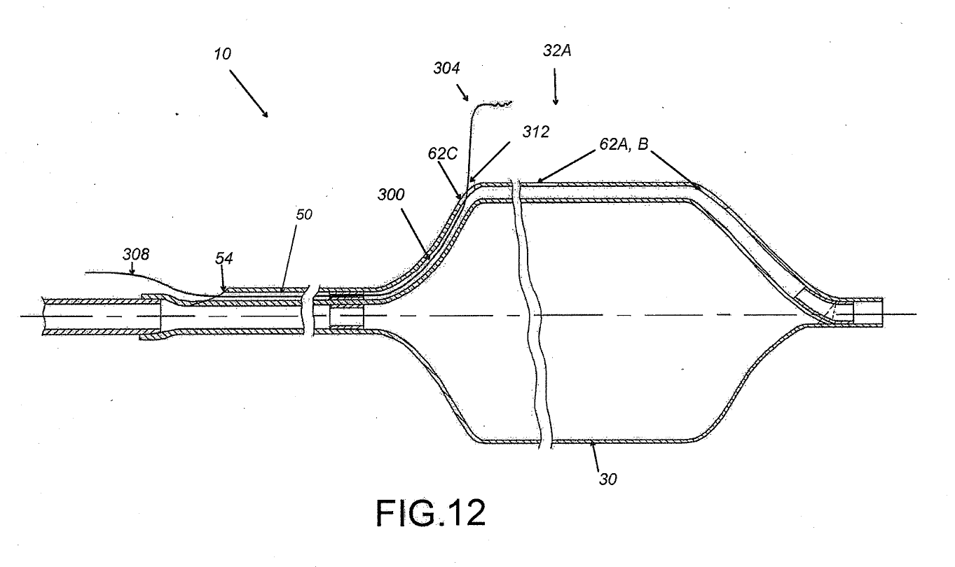

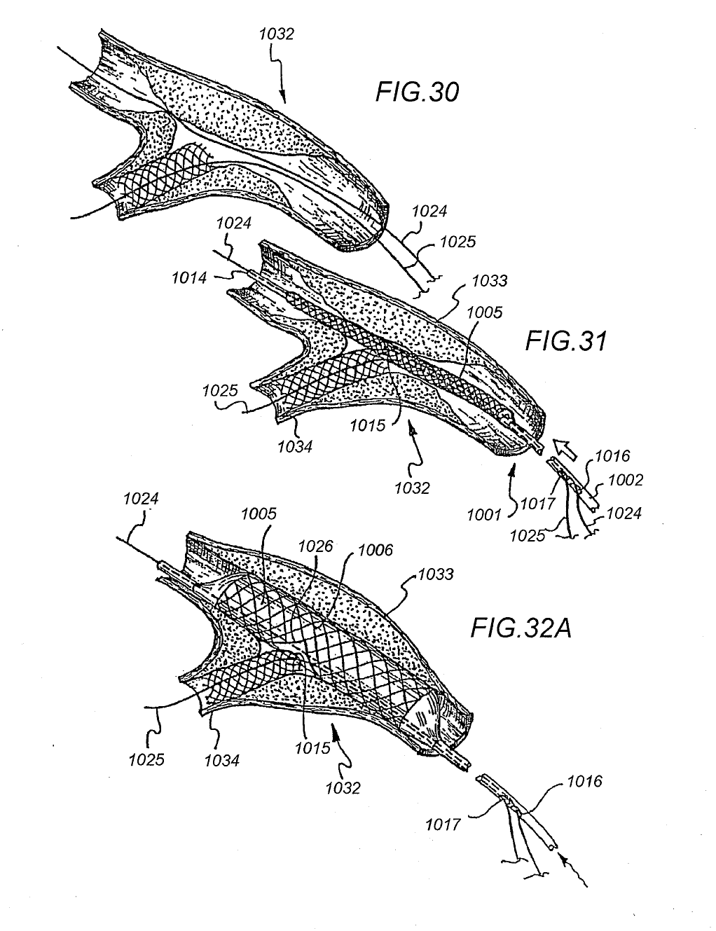

The distal portion 22 of the catheter assembly 10 is configured for performing a treatment of a lesion within the vasculature. The catheter assembly 10 is particularly suited for treating lesions or blockages adjacent to vascular bifurcations, including ostial bifurcations, Y-bifurcations, and T-bifurcations. Techniques for treating patients using the catheter assembly 10 and other endolumenal devices described herein are discussed further below in connection with FIGS. 12-14F, 27-37, and 55-70, discussed further below.

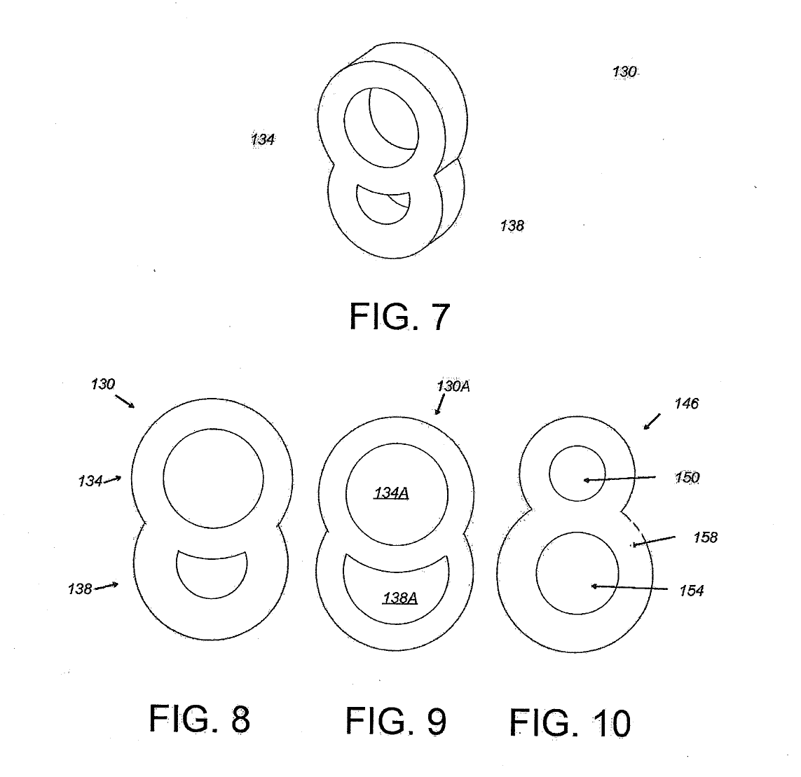

In one embodiment, the distal portion 22 includes an expansion device 30 that is configured to be expandable into engagement with an inner surface of a body lumen, such as a blood vessel. The expansion device 30 can be formed using any suitable technique, such as those discussed below in connection with FIGS. 7-10. In certain embodiments, the expansion device 30 is configured to have mounted thereon an endovascular prosthesis, such as a stent. Positioning of a stent on the expansion device 30 is discussed further below in connection with FIGS. 12-14F, 27-37, and 55-70.

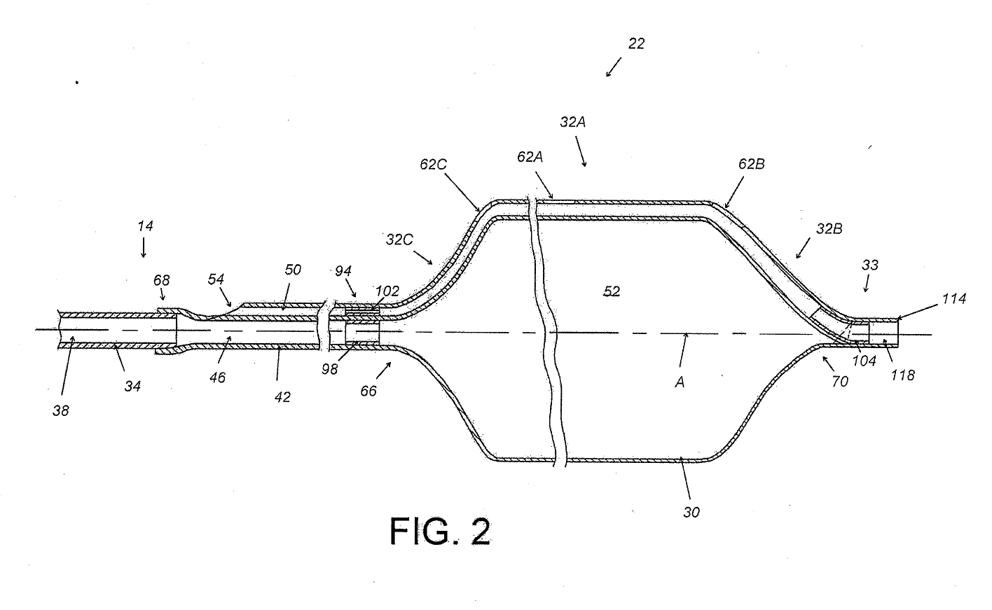

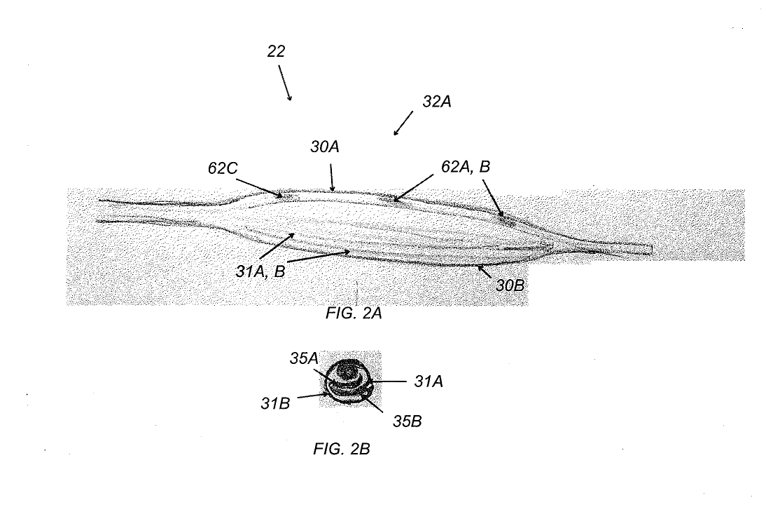

FIG. 2 shows that the expansion device 30 can include a longitudinally extending central portion 32A, a tapered distal portion 32B, and a proximal tapered portion 32C. The central portion 32A is configured to be expanded from a low profile configuration (in transverse cross-section) in which a length of the central portion 32A is adjacent to a central longitudinal axis A of the catheter assembly 10 to an enlarge configuration (in cross-section) in which the length of the central portion 32A is disposed away from the longitudinal axis A. The low profile configuration can take any suitable form, such as being folded or pleated as illustrated in FIGS. 2A-2B, discussed further below. In one embodiment, the expansion device 30 comprises a generally cylindrical or tubular surface in the second configuration that is larger than the surface of the central portion 32A in the first configuration. In some embodiments, the expansion of the expansion device 30 is generally symmetrical. In one generally symmetrical arrangement, the distance from the axis A to the outer surface of the balloon within the central portion 32A adjacent is substantially equal around the perimeter of the expansion device. In one generally symmetrical arrangement, the distance from the axis A to the outer surface of the expansion device 30 within the central portion 32A is substantially equal along the length of the central portion 32A.

The distal tapered portion 32B of the expansion device 30 provides a progressively enlarged outer surface from distal to proximal at least when the expansion device 30 is expanded. In one arrangement, the distal tapered portion 32B is arranged such that the outer circumference of the expansion device 30 increases from being substantially equal to an outer circumference of a tip portion 33 of the catheter assembly 10 to being substantially equal to the outer circumference of the expansion device 30 at the central portion 32A. The distal tapered portion 32B preferably is configured to enhance advancement of the catheter assembly 10 through the vasculature of a patient. For example, where the patient's vessel is at least partially occluded by a lesion, the distal tapered portion 32B can provide a means for easing the distal portion 22 through the lesion.

The proximal tapered portion 32C can also be provided to facilitate proximal movement through the vasculature, e.g., during removal or retraction. For example, in some techniques a treatment may cause plaque to shift into the vascular lumen in which the expansion device 30 is disposed. Such proximally located plaque or lesion may need to be traversed by the distal portion 22 of the catheter assembly 10 for further treatment or for removal of the catheter assembly 10. Accordingly, by providing a tapered profile during such movement enables the distal portion 22 of be retracted.

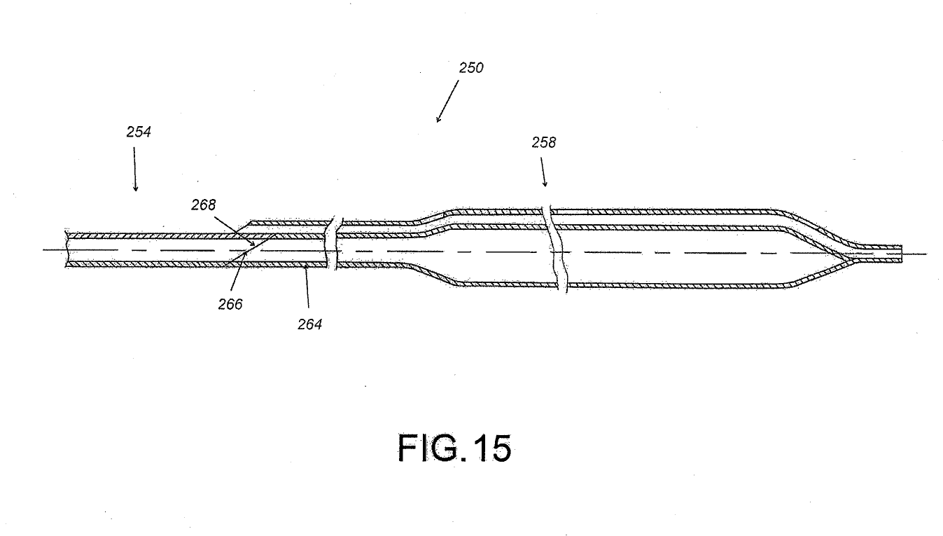

The catheter assembly 10 can be configured for any treatment, but is particularly useful for treatment of certain blood vessels that are very small. For example the catheter assembly 10 can be used to treat coronary vessels, cerebral vessels, and other very small vessels that are difficult to reach using conventional percutaneous techniques. In some embodiments disclosed herein, the catheter assembly is formed with fewer features than a conventional catheter, which features tend to enlarge the profile of the catheter. For example, catheter assemblies are described in connection with FIGS. 1, 2, 12-14, and 15 that have only a single guidewire lumen but which are still configured to enable precise positioning at a vascular bifurcation. Also, in connection with FIG. 15, embodiments are discussed in which fewer joints or points of connection than is conventional are provided. By reducing the number of components, the overall profile of the device can be reduced.

The catheter assembly 10 is advantageous in that by eliminating an inner pipe or other lumen defining structure within the balloon, e.g., along the axis A, at least the distal portion 22 of the assembly 10 can be reduced in size. By reducing the size or bulkinesss of the distal portion 22, the catheter assembly 10 can be used in very small vessels and bifurcation locations. The device 10 can also be used with a plurality of guidewires to enable placement of the expansion device 30 a small vessel bifucations. This construction enables placement at in challenging procedures, such as where only very small passage remains in the vessel, e.g., total or near total occlusion procedures.

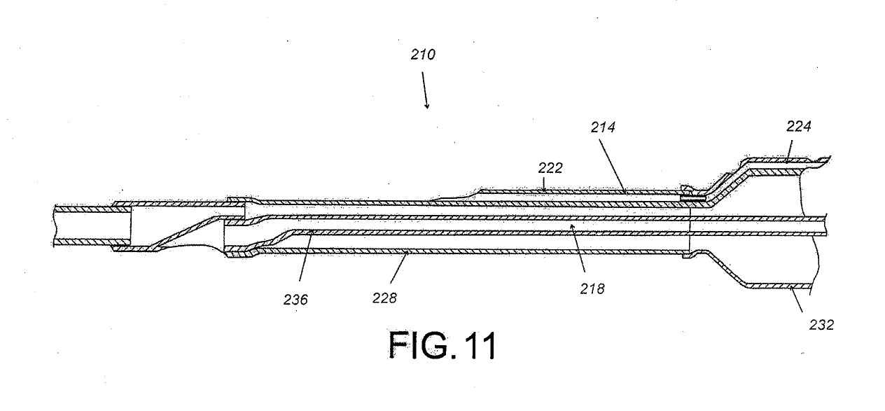

Furthermore, FIG. 11 illustrate embodiments where multiple guidewire lumens are provided but which are still configured for use in very small vessels because by extending a portion of a second guidewire lumen through a multilumen neck, a reduction in overall size is achieved, leaving more space for an inflating lumen.

In some embodiments, an elongate member 34 is disposed between the connector 26 and the distal portion 22 that is configured to convey inflation media from the proximal end of the catheter assembly 10 to the expansion device 30. In particular, the elongate member 34 can include a lumen 38 disposed therein in fluid communication with the connector 26 and in fluid communication with the expansion device 30 in the distal portion 22. In one embodiment, the elongate member 34 comprises a hypotube formed of a suitable material, such as steel, or a suitable alloy thereof, a nickel-titanium alloy or any other material that exhibit sufficient rigidity to be punishable within the vasculature in a percutaneous procedure.

The distal portion 22 of the catheter assembly 10 preferably includes an elongated neck 42 disposed between the elongate member 34 and the expansion device 30. The elongated neck 42 preferably is configured with an inflation lumen 46 and at least one auxiliary lumen 50. In one embodiment the elongated neck 42 comprises two lumens formed in a single piece of material, e.g., the inflation lumen 46 and a guidewire lumen 50. The elongated neck 42 can be formed any suitable method. In another embodiment illustrated below in FIG. 15, the elongated neck is eliminated and instead a proximally extending lumen structure similar to the elongate member 34 is directly coupled with a lumen structure of an expansion device. Preferably a majority of the cross-sectional area of the elongated neck 42 is dedicated to the inflation lumen 36 and a minority of the cross-sectional area is dedicated to be auxiliary lumen 50. One advantage of the catheter assembly 10 is that in the elongated neck 42, the lumens 46, 50 are formed in a single, monolithic structure. For example, a continuous wall structure is provided that surrounds and both of the lumens 46, 50. In some embodiments, a continuous wall structure extends from luminal surfaces defining both of the lumens 46, 50 to an external surfaces of the elongated neck 42.

The inflation lumen 46 provides fluid communication between the lumen 38 and a cavity 52 formed within the expansion device 30 in one embodiment. The lumen 46 is also in fluid communication with a source of inflation media connected to the connector 26 by way of the lumen 38 of the elongate body 14. As such, the lumen 48 facilitates expansion of the expansion device 30 in a procedure, as discussed below.

Where the catheter assembly 10 is to be used within small vessels located deep inside a patient, the distal portion 22 should be very flexible. Such flexibility can be provided, for example, by forming the elongated neck 42 of material that is very flexible or bendable. Such bendability enables the distal portion 22 to follow a guidewire around a highly tortuous, curving path through the vasculature. Preferably the elongated neck 42 is relatively long, such as for example extending it least about 6 cm in length. In one embodiment the elongated neck 42 extends up to about 20 cm in length. In another embodiment, the elongated neck 42 extends anywhere between about 6 cm and about 20 cm in length.

The auxiliary lumen 50 can be configured as a guidewire lumen in one embodiment. FIGS. 12-14F illustrate the use of the lumen 50 in connection with one or more guidewires. The guidewire lumen 50 can form a portion of a guidewire tracking device. In various embodiments, guidewire tracking devices can include one or a plurality of guidewire lumens and one or a plurality of distal ports as discussed further below. Distal ports can be located in any convenient location, such as along the side of the expansion device 30 or at an apical location (e.g., a distal-most location on the elongate body 14). The guidewire lumen 50 preferably includes a proximal guidewire port 54 and a positioning device 58 located distal of the proximal guidewire port 54. The positioning device 58 preferably includes a plurality of distal ports 62 located distal of the proximal guidewire port 54.

In one embodiment, the positioning device 58 includes three distal ports 62A, 62B, 62C located between proximal and distal ends 66, 70 of the expansion device 30. The distal ports 62A-62C can be disposed in any suitable location for assisting in positioning the expansion device 30 relative to certain vascular anatomy, as discussed further below.

In one embodiment, a proximal-most distal port 62C is located between the proximal end 66 of the expansion device 30 and a proximal end of a longitudinally extending active portion. The proximal-most distal port 62C is located between the proximal end 66 and the central portion 32A of the expansion device 30. The port 62C can be located at the junction of the proximal tapered portion 32C and the central portion 32A of the expansion device 30.

In one embodiment, a central distal port 62A is located within a longitudinally extending active portion, e.g., the central portion 32A of the expansion device 30. The port 62A can be located generally centrally in the central portion 32A or can be disposed closer to the proximal or distal ends thereof. Preferably at least one of the ports 62A, 62B, 62C is located such that sufficient length of the central portion 32A is provided to mount a vascular prosthesis such as a stent between the port and an adjacent port. For example, the port 62A can be located such that sufficient length of the central portion 32A is provided to mount a vascular prosthesis between the port 62A and the port 62B, which would facilitate the treatment illustrated in FIGS. 12-14F, 27-37, and 55-70.

The port 62A can be located such that sufficient length of the central portion 32A is provided to mount a vascular prosthesis between the port 62A and the port 62C, which would facilitate the treatment illustrated in FIGS. 12-14F, 27-37, and 55-70.

In one embodiment, a distal-most distal port 62B is located between the distal end 70 of the expansion device 30 and a distal end of the central portion 32A of the expansion device 30. The port 62C can be located at the junction of the distal tapered portion 32B and the central portion 32A.

In one embodiment, a joint 68 is provided between the elongate member 34 and the elongated neck 42. The joint 68 can take any suitable form, such as for example, providing a direct connection between the elongate member 34 and the elongated neck 42. FIG. 2 shows that in one embodiment a connection can be made between the elongate member 34 and the elongated neck 42 by inserting one into the other. In one arrangement, a distal end of the elongate member 34 is positioned distal of a proximal end of the elongated neck 42, for example being received at least partially in the inflation lumen 46. In one arrangement, the elongate member 34 is relatively rigid and the elongated neck 42 is at least somewhat deformable such that a proximal end of the elongated neck 42 can be enlarged to receive a distal end portion of the elongate member 34. The joint 68 can be further secured by any conventional means.

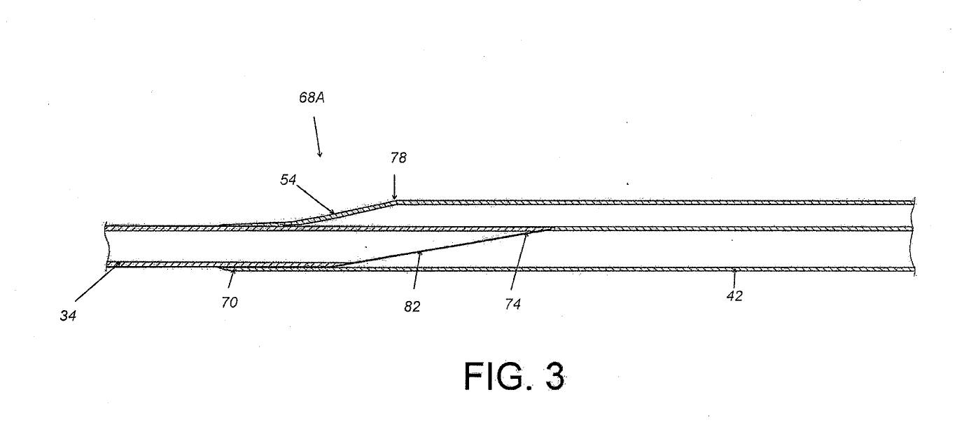

FIG. 3 shows another embodiment of a joint 68A arranged to reinforce a proximal portion of the elongated neck 42. In particular, as discussed above, the elongated neck 42 includes a proximal guide wire port 54. The guide wire port 54 preferably is disposed adjacent a proximal end 70 of the elongated neck 42. In some embodiments, it is preferable to stiffen the region of the elongated neck 42 adjacent to the proximal guide wire port 54. Such stiffening provides advantages, such as reducing the amount of bending at the joint 68A due to the insertion or presence of a guide wire within the auxiliary lumen 50. In one technique the region adjacent to the guide wire port 54 can be stiffened by extending a portion of the elongate member 34 into the inflation lumen 46. For example, a portion of the elongate member 34 can be inserted distally into the inflation lumen 46 until a distal end 74 of the elongate member 34 is distal of the distal end 78 of the proximal guide wire port 54. In this position, a portion of the elongate member 34 is located at the interface between the inflation and auxiliary lumens 46, 50. In various embodiments, the elongate member 34 is much stiffer than the elongated neck 42. Thus, the presence of a portion of the elongate member 34 at the interface between the inflation and auxiliary lumens 46, 50 greatly increases the stiffness of the elongate body 14 at this interface. This arrangement is advantageous in that it enhances the ability of the elongated neck 42 to absorb the concentrated lateral forces that can result when a guidewire is disposed in the region adjacent to the proximal guide wire port 54.

As discussed further in connection with FIG. 15, some embodiment are further modified such that a direct end-to-end connection is provided between two separable component of a catheter assembly. For example, an end-to-end connection can be provided between a hypotube or other elongate member and an elongated neck or other distal portion of a catheter assembly.

FIGS. 1 and 11 illustrate that in some embodiments, an intermediate shaft can be included in a catheter assembly between a proximal luminal structure such as a hypotube and a distal luminal structure. In FIG. 1, an intermediate member 69 is provided between the elongate member 34 and the elongated neck 42. The intermediate member 69 provides for increasing the length of the catheter assembly 10 in some embodiments. In some arrangements, the intermediate member 69 can provide a transition in flexibility such as from a relatively stiff hypotube to a relatively flexible expansion member.

FIG. 3 illustrates that the elongate member 34 can be fluted at the distal end thereof in some embodiments. For example, the distal end of the elongate member 34 can comprise a tapered end 82 in which a first side thereof adjacent to the auxiliary lumen 50 can extend further distally than a second side thereof that is not adjacent to the auxiliary lumen 50. The tapered end 82 can be configured such that when the elongate member 34 is inserted into the elongated neck 42 a distal portion of the tapered end 82 is distal of the guide wire port 54 and a proximal end of the tapered end 82 is proximal of at least a portion of the guide wire port 54. By providing the tapered end 82 on the elongate member 34, the joint 68A is optimized such that reinforcement is provided adjacent to the guide wire port 54 but the flexibility of the elongated neck 42 is maximized so that the tractability of the catheter assembly 10 is maximized.

In one embodiment the elongated neck 42 is formed of a Polyether block amide or other suitable thermoplastic elastomer. One manufacturer offers this material under the trade name PEBAX®. Other suitable materials include variations of polyamides, such as Nylon 12.

In one embodiment the expansion device 30 and the elongated neck 42 are coupled. For example, the expansion device 30 and the elongated neck 42 can be directly coupled together at a joint 94. Any suitable technique can be used to join the elongated neck 42 to the expansion device 30. The joint 94 preferably is located at or adjacent to the proximal end 66 of the expansion device 30. Preferably a distal portion of the elongated neck 42 and a proximal portion of the expansion device 30 comprise substantially similar configurations. For example, the proximal portion of the expansion device 30 can have substantially the same shape as a distal portion of the elongated neck 42. These arrangements permit an outer profile, e.g., circumference or perimeter, of the catheter assembly 10 to be substantially constant on both proximal and distal sides of the joint 94. Any suitable technique can be used to secure the proximal portion of the expansion device 30 to the distal portion of the elongated neck 42.

Preferably, a radiopaque marker is provided in the region of the joint 94. In one technique, the radiopaque marker is formed by embedding a sufficient quantity of a radiopaque substance within a wall structure of at least one of the elongated neck 42 and the expansion device 30 adjacent the joint 94. For example, in forming the elongated neck 42, a powder of the materials such as gold can be mixed with the material of which the elongated neck 42 is to be formed. When the elongated neck 42 is formed, the metallic powder will be embedded in the wall structure.

In one embodiment, a similar technique can be used to embed a metal powder in a proximal portion of the expansion device 30. In another embodiment, a metal powder is embedded both in a distal portion of the elongated neck 42 and in a proximal portion of the expansion device 30.

In an alternative embodiment, a radiopaque marker is provided by inserting a tubular member 98 within the lumen 46 of the elongated neck 42, within a lumen of the proximal portion 66 of the expansion device 30, or both within the lumen 46 of the elongated neck 42 and within a lumen of a proximal portion 66 of the expansion device 30.

In another embodiment, a radiopaque marker is provided by positioning a tubular member 102 with in the auxiliary lumen 50 of the elongated neck 42, within a proximal portion 66 of an auxiliary lumen of the expansion device 30, or within both the auxiliary lumen 50 and a lumen of the expansion device 30. In one additional embodiment, tubular members 98, 102 are positioned in both a main inflation lumen and an auxiliary lumen of the catheter assembly 10.

For some embodiments, it is also desirable to be able to locate and/or track the position of a distal portion other expansion device. Accordingly, in some embodiments, a radiopaque tubular member 104 is provided in the distal portion 22. The tubular member 104 can be located at the distal end of the expansion device 30, for example. Any other suitable technique can be used to provide a radiopaque marker at the distal portion 22, e.g., embedding a metallic powder within a wall of the distal portion at the distal end of the expansion device 30.

The expansion device 30 preferably is configured to be used in a percutaneous procedure to treat a patient. For example, the expansion device 30 can be optimized for expanding to compress a lesion within a vasculature, such as at or adjacent to and vascular bifurcation. The expansion device 30 can be optimized to deploy a stent at a lesion within a vasculature, such as at or adjacent to and vascular bifurcation.

In one embodiment, the expansion device 30 comprises a generally noncompliant material, such as a suitable polyamide, for example nylon 12. As discussed above, preferably the expansion device 30 includes an expandable portion that can be expanded from a low-profile condition to a higher profile condition. The low-profile condition facilitating advancement of the catheter assembly 10 through the vasculature and the high profile condition facilitates performing one or more treatments. In one embodiment, the low-profile condition is suitable for delivering a stent or other endolumenal prosthesis and the high profile condition is suitable for deploying the stent or other endolumenal prosthesis. Preferably the expandable portion of the expansion device 30 is configured to expand generally symmetrically with respect to a central longitudinal axis A.

In various embodiments, the entire circumferential surface of the expandable portion of the expansion device 30 moves from the low-profile condition to the high profile condition during expansion of the expandable portion. Such movement causes the entire circumferential surface of the central portion 32A to move away from the central longitudinal axis A during such expansion, as discussed above.

Although the unexpanded or low profile configuration can take any suitable form, FIGS. 2A-2B illustrate an unexpanded configuration of the distal portion 22 that is configured to reduce asymmetry of the distal portion 22. Asymmetry can be introduced by the relative position of components of the distal portion 22. For example, as discussed further below a guidewire lumen preferably is positioned within a wall structure of the expansion device 30. Preferably the lumen disposed within the wall structure is the only guidewire lumen at that location at least along the central portion 32A of the balloon. By minimizing the asymmetry, the catheter assembly 10 can be more easily moved through body conduit, such as blood vessels. One technique for minimizing the asymmetry of the distal portion 22 due to this peripheral positioning of the guidewire lumen is to fold the rest of the expandable wall structure of the expansion device around the portion of the wall structure containing the peripherally located guidewire lumen.

FIG. 2B also illustrates an advantageous feature of various embodiments in that the distal portion 22 of the device 10 includes only a single guidewire passage. Other existing devices have multiple guidewire passages in distal segments, which results in a bulky arrangement that is not suitable for very small vessels. Also, because the interior of the balloon is without guidewire tubes or other stiffeners, the balloon can be folded about the single lumen 50 and still present a compact cylindrical profile in cross-section, as shown in FIG. 2A. For example, one specific prior balloon design provided a transverse diameter of 0.8 mm or more. However, various embodiments herein can provide diameters smaller than 0.8 mm. For example 0.60 mm or less than 0.60 mm. Some embodiments provide a transverse diameter of about 0.55 mm or less. Other embodiments provide a low profile configuration, where prior art designs provided a low profile configuration that is about 40% to about 50% larger than that of embodiments similar to FIG. 1-2B. Thus, the delivery dimensions of balloon catheter can be substantially smaller than conventional, prior art balloon catheters.

More particularly, FIG. 2A shows that a first lateral portion 30A of the expansion device 30 corresponds to a portion of the wall of the expansion device through which various ports 62A, 62B, 62C can be located. A second lateral portion 30B of the expansion device is disposed on the opposite side of the longitudinal axis A of the catheter assembly 10. The second lateral portion 30B includes all of the outer surface area of the expansion device 30, other than that corresponding to the first lateral portion 30A.

In one embodiment, the second lateral portion 30B can be arranged into a plurality of folds or pleats 31A, 31B that are arranged to minimize the cross-sectional profile of the expansion device 30. The pleats 31A, 31B can be arranged to enhance symmetry of the expansion device 30 at least in the central portion 32A. For example, the pleat 31A can comprise a concave portion 35A that is configured to receive a convex portion of the first lateral portion 30A of the expansion device 30. Also, the pleat 31B can comprise a concave portion 35B that is configured to receive a convex portion of the pleat 31A. This arrangement can result in a substantially circular outer perimeter of the expansion device 30 at least in the central portion 32A, as illustrated by FIG. 2B. In some embodiments, more than two pleats can be provided to maintain a compact structure while enhancing symmetry in an unexpanded state.

In one embodiment, the auxiliary lumen 50 of the catheter assembly 10 extends from within the elongated neck 42 to within the expansion device 30. Various embodiments the auxiliary lumen 50 extends from the proximal port 54 to a distal, apical port 114. The proximal port 54 can be located anywhere along the elongate body 14 of the catheter assembly 10. Preferably, the proximal port 54 is located distal of the joint 68 between the elongate member 34 and the elongated neck 42. These locations provide for rapid exchange, for example rapid mounting of the catheter assembly 10 onto the guidewire after removal of another device from the guidewire.

FIG. 2 shows additional details of the distal portion 22 of the catheter assembly 10. For example, the auxiliary lumen 50 extends along the expansion device 30 through the distal tapered portion 32B to the distal, apical port 114. More particularly, the auxiliary lumen 50 includes a distal portion 118 located distal of the distal tapered portion 32B. The distal portion 118 of the auxiliary lumen 50 preferably extends longitudinally, for example along the axis A. As discussed further below, this arrangement of the auxiliary lumen 50 enables a guidewire to be disposed within the auxiliary lumen 50 such that the guidewire extends from the proximal guide wire port 54 through the proximal tapered portion 32C, the central portion 32A, the distal tapered portion 32B in along the distal portion 118 of the auxiliary lumen 50, along the axis A.

In various techniques, a distal end of the guidewire can be positioned relative to the catheter assembly 10 such that relative movement therebetween can cause the distal end of the guidewire to protrude out of any of the distal, apical port 114, any of the side ports 62A-62C, or to be withdrawn from the proximal port 54. Various methods of treatment in accordance with these techniques are discussed below in connection with FIGS. 12-14F, 27-37, and 55-70.

FIG. 7-10 illustrate various techniques for forming a portion of a catheter assembly. The techniques corresponding to FIGS. 7-10 are particularly useful for forming lumenal structures, e.g., where multiple lumens are arranged within a single wall structure. These techniques can be used to form, for example, the expansion device 30 or the elongated neck 42 and corresponding lumens. In one embodiment, first and second portion of the inflation lumen 46 extend within the elongate neck 42 and expansion device 30 respectively. In one embodiment, first and second portion of the auxiliary lumen 50 extend within the elongate neck 42 and expansion device 30 respectively.

FIGS. 7-10 illustrate a cross-sectional portion of a preformed construct 130 that is provided with a guidewire portion 134 and inflation portion 138. In a subsequent step, the construct 130 can be processed into the expansion device 30, for example. The guidewire portion 134 in the preformed construct 130 can be configured with a shape suitable for use in connection with a guidewire. For example, the guidewire portion 134 in the preformed construct 130 can have a size and shape suitable for receiving a guide wire. In one embodiment, the guidewire portion 134 comprises a substantially circular cross-section which is sized to selectively receive a guide wire. In one embodiment the inflation portion 138 of the preformed construct 130 defines a relatively small lumen that is expanded during a subsequent manufacturing process to form the inflating chamber or cavity 50 of the expansion device 30.

In the first technique, the guidewire portion 134 is inserted over a mandrel (not shown) that is configured to reside within the guidewire portion 134 to substantially maintain the shape of the guidewire portion 134 during the formation of the inflation portion 138. The inflation portion 138 can be formed in any suitable way, such as using cold deformation of the preformed construct 130 inside a mold. In addition, the inflatable portion 138 can be formed into an expansion device 30 by enlarging the portion 138 into the cavity 52. This process of formation will cause the inflation portion 138 to be expanded to provide the shape of the expandable device 30.

FIG. 9 illustrates another embodiment in which a preformed construct 130A is configured with a larger inflation portion including a lumen 138A that is larger than the lumen of the inflation portion 138. Enlarging the lumens 138A can be achieved by any suitable manner, such as by making the wall of the construct 130A thinner than that of the construct 130. Also, because the construct 130A is thinner, the process related to FIG. 9 can produce a thinner expansion device after the process is complete. By making the expansion device thinner, a greater variety of vessels can be treated.

FIG. 10 illustrates a preformed construct 146 that is suitable for formation of another embodiment of the distal portion 22. The preformed construct 146 preferably includes first and second lumens 150, 154. The lumens 150, 154 preferably are disposed within a wall structure 158 this substantially continuous. In particular, the wall structure 158 preferably is a unitary structure and is not formed by attaching multiple elongate bodies together. The wall structure 158 can be formed by a single mold process using a single material. This enables the formation of an expansion device that has a monolithic construction. This construction provides many advantages, including making the overall catheter assembly more able to access and treat very small vessels, e.g., by being lower profile and/or less flexible.

Formation of the distal portion 22 of the preformed construct 146 can be achieved in any suitable technique. For example in one embodiment, the first lumen 150 can be formed into the auxiliary lumen 50. In one embodiment the second lumens 154 can be formed into the inflation lumen 46 or into the cavity 52 of the expansion device 30. One technique for forming the inflation lumen 46 from the second lumen 154 is to inflate the second lumen 154 under controlled conditions. For example in one technique, the preformed construct 146 is cold deformed inside a mold to expand the second lumen 154 to form an expansion chamber, such as the cavity 52. The first lumen 150 can be formed into the auxiliary lumen 50 in any suitable technique. In one approach, the first lumen 150 is expanded into the auxiliary lumen 50 by increasing the pressure within the first lumen 150 under controlled conditions. For example, the first lumen 150 can be cold deformed inside a mold to expand the size of the lumen 150. The expanded size of the first lumen 150 can be any suitable size, but preferably is such that the expanded size can accommodate a guide wire, as discussed above in connection with the auxiliary lumen 50.

FIG. 11 shows another embodiment of the catheter assembly 210 that is similar to the catheter assembly 10 except as set forth below. The catheter assembly 210 includes two distinct guidewire lumens, a first guidewire lumen 214, and a second guidewire lumen 218. The first guidewire lumen 214 is similar to the auxiliary lumen 50, and includes a proximal portion 222 and a distal portion 224. The proximal portion 222 is formed in an elongated neck 228 and the distal portion 224 is formed in an expandable portion 232. The second guidewire lumen 218 is formed within a separate elongate member 236. The elongate member 236 extends within an inflation lumen 240 of the elongated neck 228. The second guidewire lumen 218 has a proximal port located approximately at the proximal end of the elongate neck 228. The first guidewire lumen 214 has a proximal port that is located distal of the proximal port of the second guidewire lumen 218. The proximal ports of the guidewire lumens 214, 218 are located on opposite sides of the elongated neck portion 222.

FIG. 15 shows a catheter assembly 250 that is similar to the catheter assembly 10 except as set forth below. For example, the catheter assembly 250 differs in that an elongate member 254 is directly coupled with a proximal end of a distal portion 258 of the catheter assembly. The distal portion 258 includes an expansion device 262 that has a nonexpandable proximal portion 264 that extends proximally therefrom to a proximal end 266. The proximal end 266 is directly coupled with a distal end 268 of the elongate member 254. The connection between the proximal end 266 and the distal end 268 is by a direct end-to-end connection. Preferably the proximal end 266 has a tapered configuration and the distal end 268 also has a tapered configuration. The direct end-to-end connection of the embodiment of FIG. 15 enables the catheter assembly to 250 to have a particularly low-profile configuration.

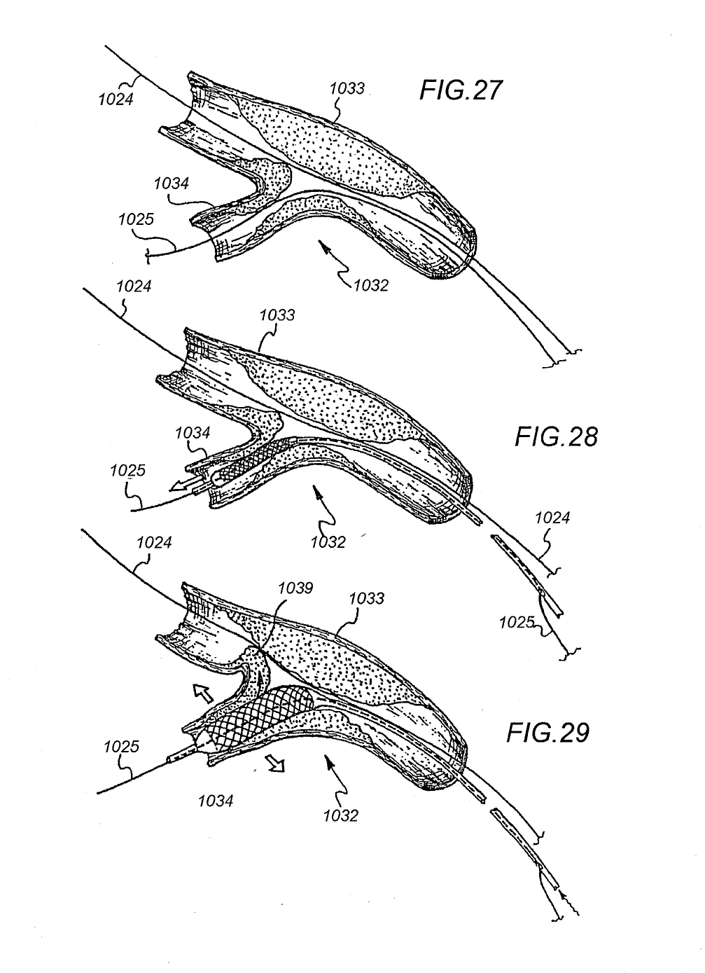

Various systems and methods of using endolumenal devices described herein will now discussed in connection with FIGS. 12-14F. Further methods related to these systems and methods are elaborated upon below in connection with FIGS. 27-38 and 55-70.

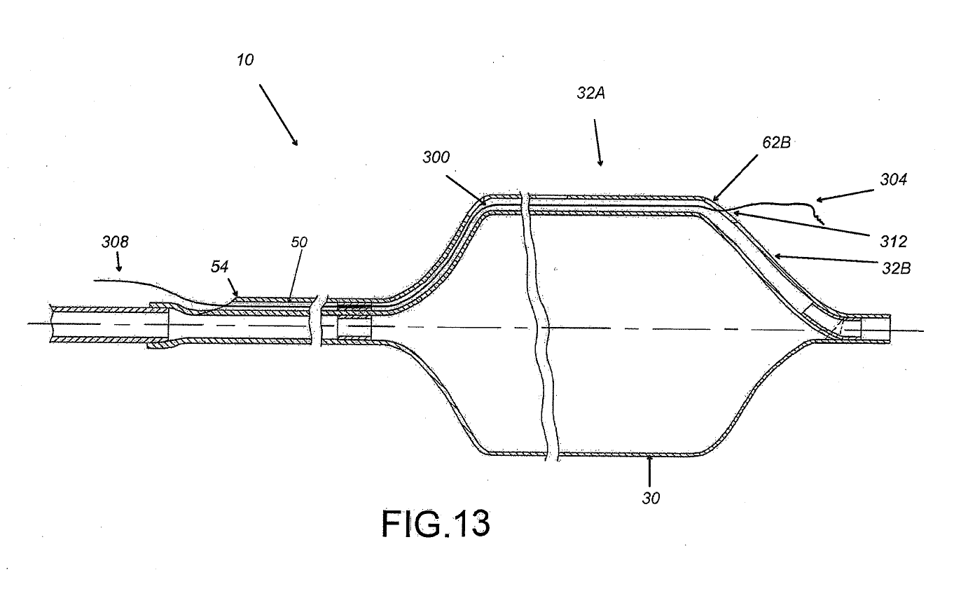

FIGS. 12 and 13 illustrate that in various techniques, the catheter assembly 10 can be used in connection with a guidewire 300 to position an active portion of the expansion device in a selected location. For example, FIG. 12 illustrates that the guidewire 300 can be advanced such that a distal portion 304 of the guidewire 300 extends through the proximal-most of the distal ports 62C. A proximal portion 308 of the guidewire 300 extends through the proximal guidewire port 54. When the guidewire 300 is positioned in this manner, the majority of the active portion of the expansion device 30 is disposed distal of the position of the distal portion 304 of the guidewire 300. A divergence 312 between the guidewire distal portion 304 and the central portion 32A of the expansion device 30 limits the advancement of the catheter assembly 10 when the system of FIG. 12 is positioned at a bifurcation. Thus, the clinician can confirm that the portion of the expansion device 30 distal of the proximal-most of the distal ports 62C is just distal of (but very near) the location of the bifurcation.

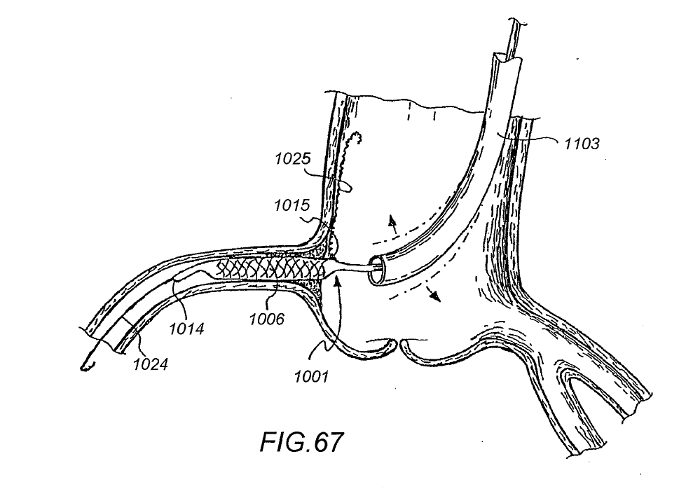

This technique is very useful in connection with a method of treating an ostium, such as the coronary ostium, as discussed further in connection with FIG. 67 below. Also, this technique is useful for treating a Y bifurcation, as illustrated in FIG. 70. The embodiments of catheters disclosed above in connection with FIGS. 1-11 and 15 can be deployed in the manner described in connection with those and related figures to advantageously treat very small vessel bifurcations.

FIG. 13 illustrates a technique that advantageously positions the central portion 32A of the expansion device 30 just proximal of a bifurcation in use. In particular, the distal portion 304 of the guidewire 300 can be positioned through the distal-most of the distal ports 62B and into a first branch or blood vessel distal of a bifurcation such that the divergence 312 is formed between the distal portion 304 and the distal tapered portion 32B of the expansion device 30. The user can urge the divergence 312 into engagement with the bifurcation to confirm proper positioning. In particular, the guidewire 300 and the catheter assembly 10 can be urged distally until the distal tapered portion contacts a vessel wall in a first branch adjacent to a bifurcation and the distal portion 304 of the guidewire 300 contacts a vessel wall in a second branch adjacent to a bifurcation. Further advancement of the catheter apparatus 10 and the guidewire 300 will cause the divergence 312 to engage the bifurcation.

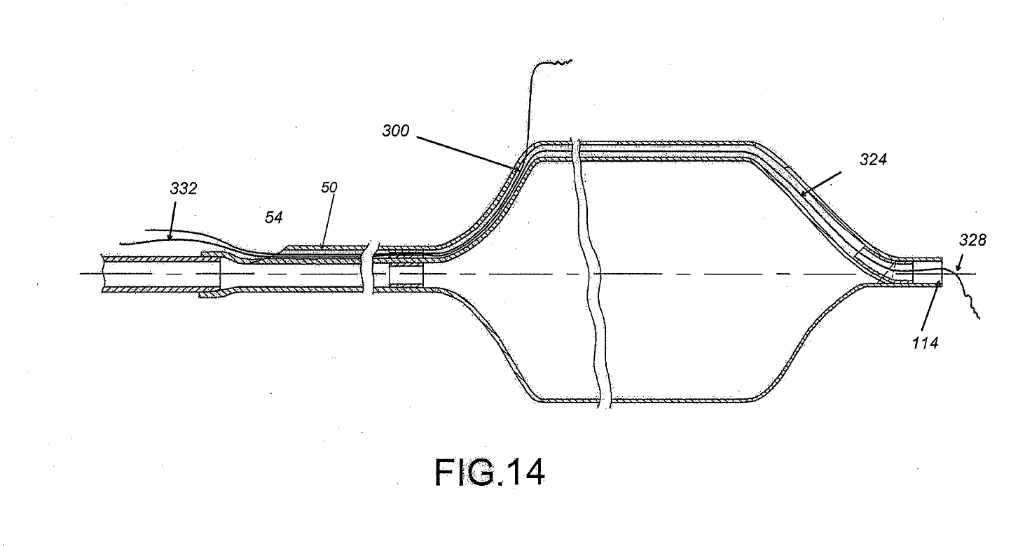

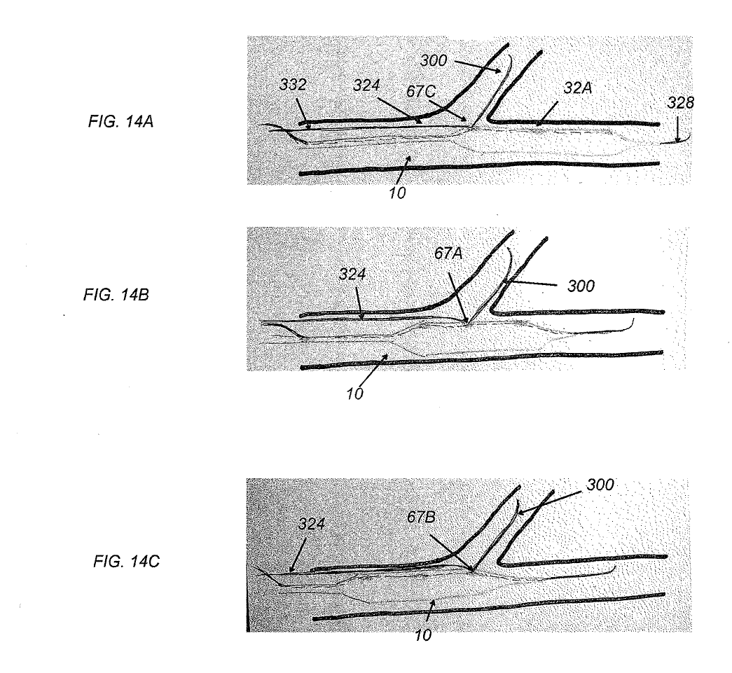

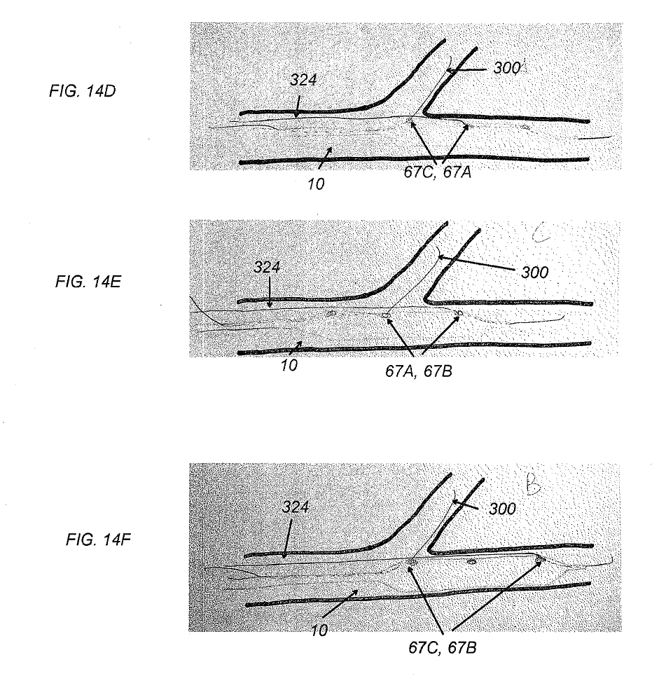

FIG. 14 illustrate another method in which multiple guidewires are used within the same lumen. This method is similar to that illustrated in FIG. 12, and also involves the use of a second guidewire 324 that can be positioned in the lumen 50 such that a distal portion 328 extends through the distal apical port 114 and a proximal portion 332 extends through the proximal port 54. The system of FIG. 14 is useful in that the second guidewire 324 provides an efficient means for tracking the catheter apparatus 10 into a distal branch vessel, e.g., a branch that is located distal of an ostium. For example, in one technique, the guidewire 324 is positioned prior to a procedure that might cause “snow-plow” or “plaque shifting” to occur. Thereafter, the catheter apparatus 10 can be advanced along the wire 324 until the distal tapered portion 32B is just proximal of the closed off vessel. Further advancement of the distal tapered portion 32B can help the catheter apparatus 10 gain access to the closed off vessel segment over the guidewire 324.

FIG. 14A illustrates another method in which multiple guidewires are used to position the catheter apparatus 10. FIG. 14A is similar to the technique described above in connection with FIG. 12 except that the guidewires 300, 324 do not overlap within the lumen 50. Rather, the proximal portion 332 of the guidewire 324 is positioned outside the catheter apparatus 10 and a distal portion of the guidewire 324 extends through the proximal most distal port 62C. As a result, the wires 300, 324 both extend through the proximal most distal port 62C. Also, the wires 300, 324 both extend within the lumen 50 but do not overlap. As discussed above, the divergence 312 between the guidewire 300 and the catheter apparatus 10 in the vicinity of the central portion 32A of the expansion device 30 provides for accurate placement of the expansion device 30 and any stent or other prosthesis associated therewith. This allows for placement of a relatively long stent distal of the bifurcation, e.g., a stent having a length about equal to the length of the central portion 32A of the expansion device 30.

FIG. 14B is similar to FIG. 14A except that the guidewires 300, 324 both extend through the central distal port 62A. As a result, a divergence 312 formed between the between the guidewire 300 and the catheter apparatus 10 forward of the central distal port 62A provides for accurate placement of a distal portion of the expansion device 30 and any stent or other prosthesis associated therewith. This allows for placement of a relatively short stent distal of the bifurcation, e.g., a stent having a length about equal to one-half the length of the central portion 32A of the expansion device 30.

FIG. 14C illustrates another method in which multiple guidewires similar to that of FIG. 14A. The proximal portion 332 of the guidewire 324 is positioned outside the catheter apparatus 10 and a distal portion of the guidewire 324 extends through the distal most distal port 62B. The wires 300, 324 both extend through the distal most distal port 62B. Also, the wires 300, 324 both extend within the lumen 50 but do not overlap. As discussed above, the divergence 312 between the guidewire 300 and the catheter apparatus 10 in the vicinity of the distal most distal port 62B of the expansion device 30 provides for accurate placement of the expansion device 30 and any stent or other prosthesis associated therewith. This allows for placement of a relatively long stent proximal of the bifurcation, e.g., a stent having a length about equal to the length of the central portion 32A of the expansion device 30. Although the wire 324 may be disposed between the stent to be deployed and the vessel wall, a suitable treatment can still be performed, such as by withdrawing the wire 324 prior to expansion or complete expansion of the expansion device for placement of the stent. In some case, the fit of the stent to the vessel wall is such that the wire may be withdrawn from between the stent and the wall without compromising the position and fit of the stent.

FIG. 14D-14F describe additional methods that can be used to place the catheter apparatus 10 and other catheter apparatuses described herein at a bifurcation. These methods are similar to those described in connection with FIGS. 14A-14C. In the methods of FIGS. 14D-14F, the guidewires 300, 324 extends through different distal ports such that the guidewires do not overlap either in the lumen 50 or at a port. In these embodiments, the guidewires 300, 324 cross each other outside of the catheter apparatus 10. These methods show that the same catheter can be used for completely different interventions. In one intervention illustrated by FIG. 14D, the wire 300 is advanced through the lumen 50 and emerges from a proximally disposed port while the wire 324 emerges from a port distal of the port from which the wire 300 emerges. In one technique a stent is place distal of the port from which the wire 324 emerges, e.g., up to and even beyond the distal most port. This enables a treatment at a position spaced distally from the port from which the wire 324 emerges by a least the inter-port spacing. In the method illustrated in FIG. 14E, the balloon can still be advanced beyond the bifurcation, but by a lesser amount. For example, about half of the balloon can be positioned proximal the bifurcation and half distal thereto. Finally, in FIG. 14F, the balloon can be positioned such that substantially the entire length of the cylindrical section is distal the bifurcation. This can enable placement of a stent distal the bifurcation that has a length of about the distance from the proximal to the distal port.

In one technique to prevent jailing the wire 324, the expansion device of the apparatus 10 can be positioned using the two wires and then the wire 324 can be withdrawn before the balloon is expanded. This provides the benefit of accurate placement without the downside of jailing the wire 324.

Thus, the same catheter design can be configured for use in three different interventions: stenosis before the bifurcation; stenosis on or at the bifurcation; and stenosis after the bifurcation, thereby providing a multipurpose catheter.

### II. Additional Catheter Apparatuses and Methods for Accessing Larger Bifurcation and Vessels

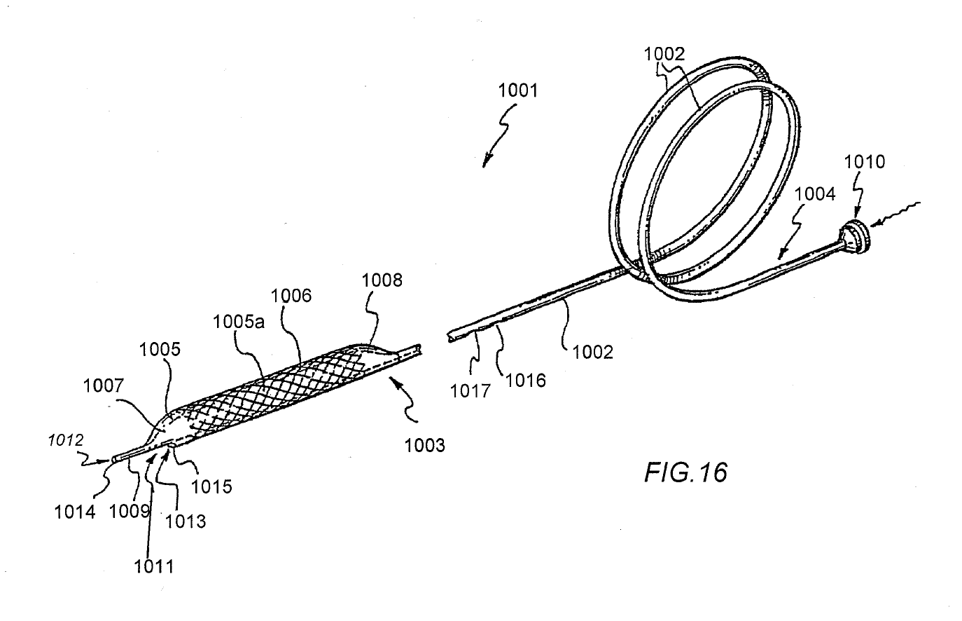

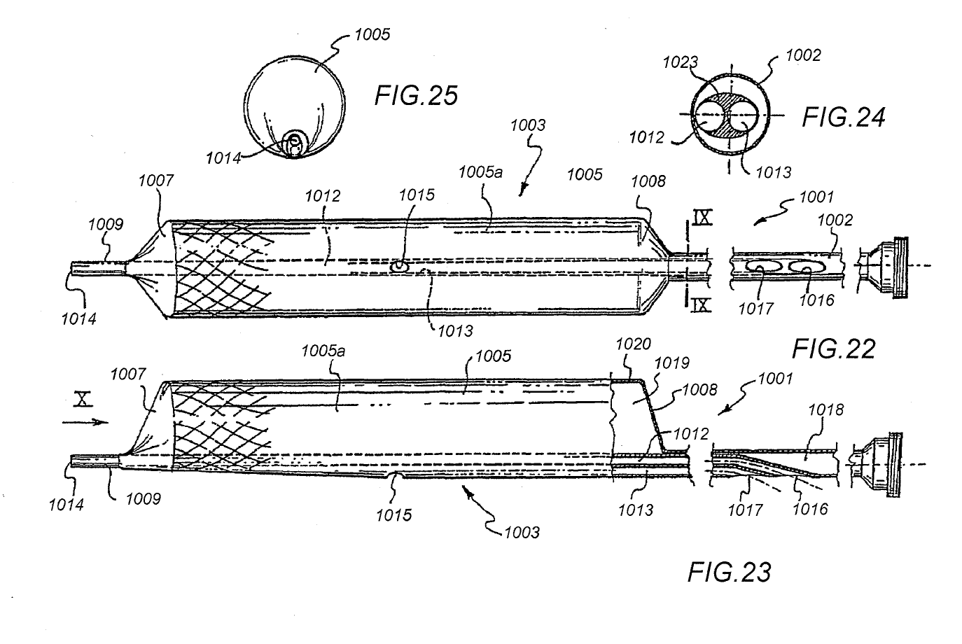

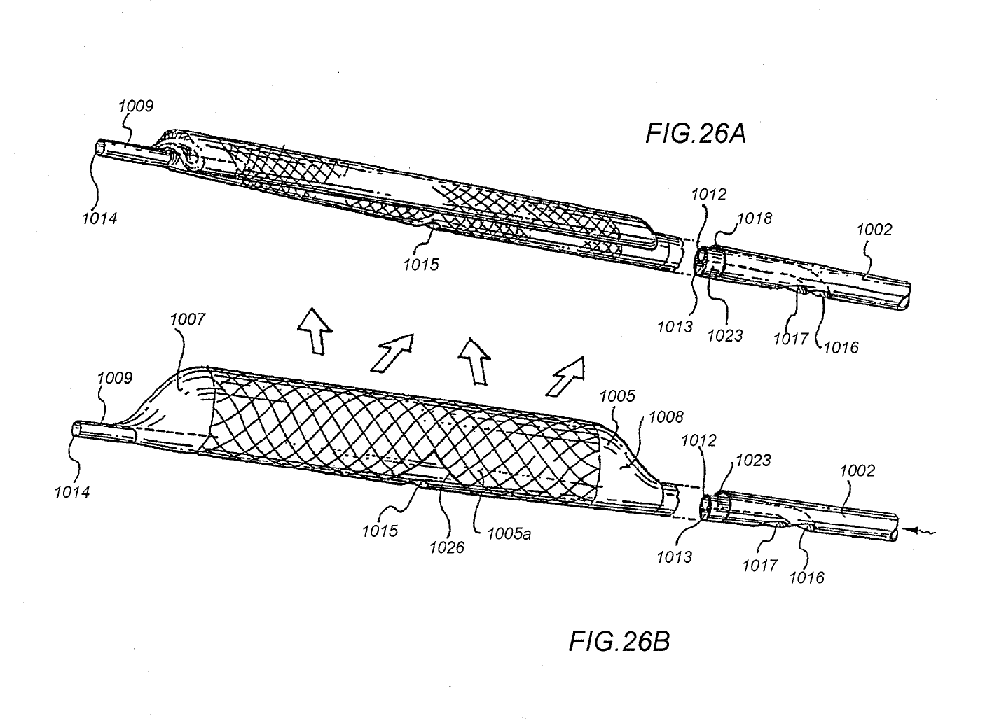

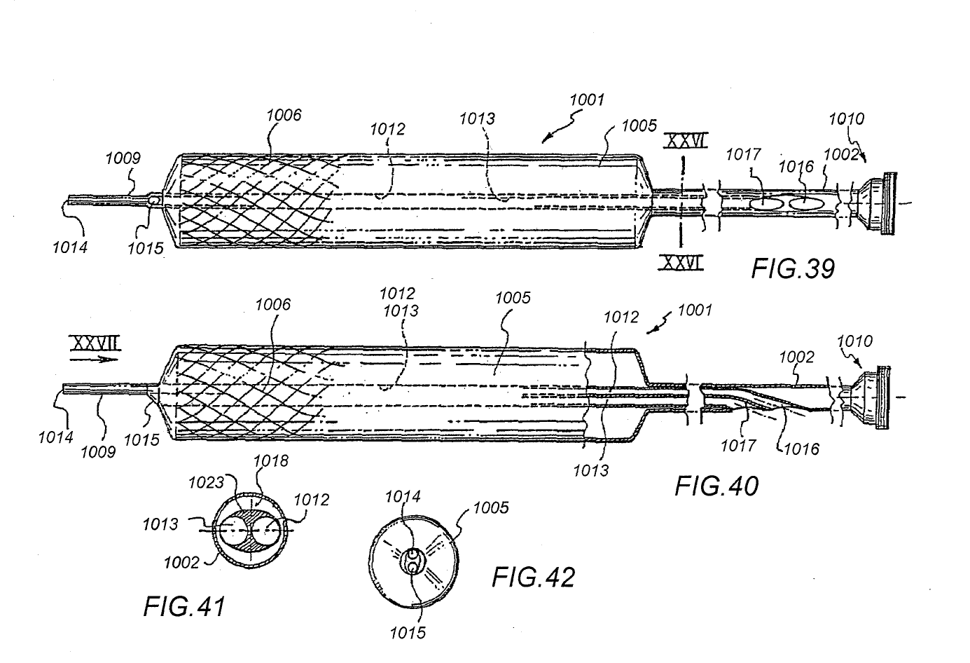

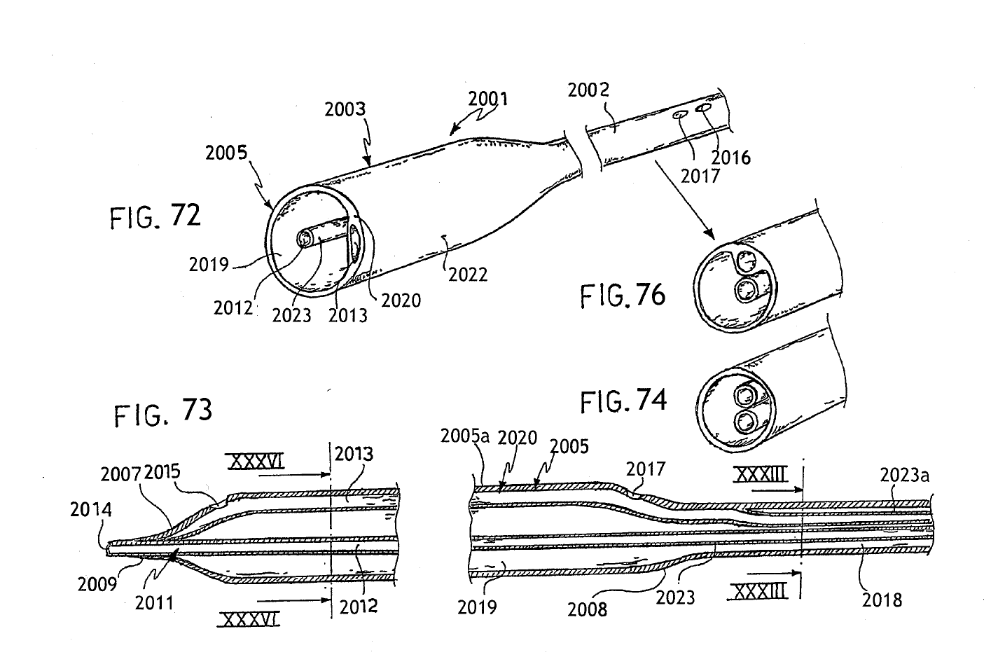

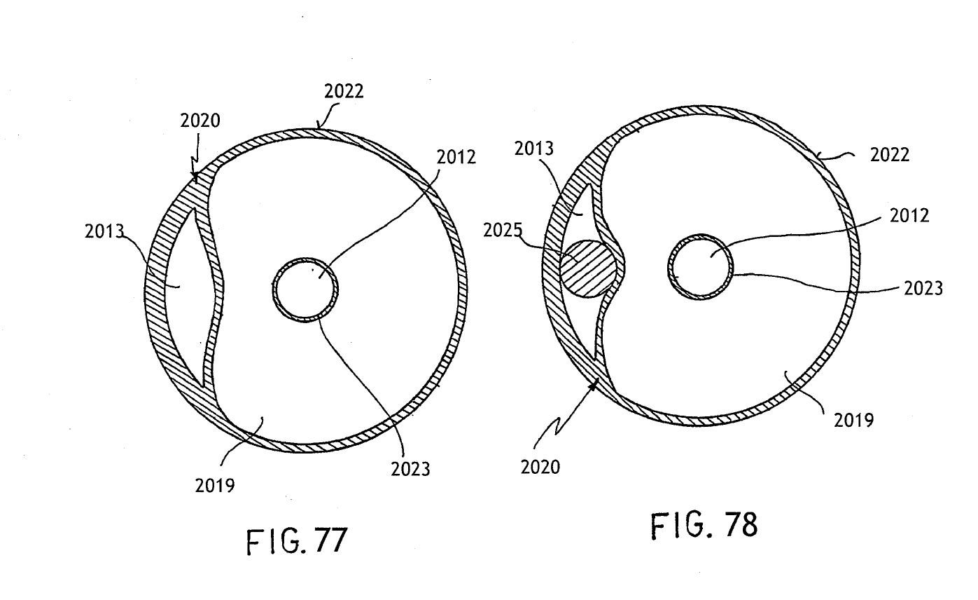

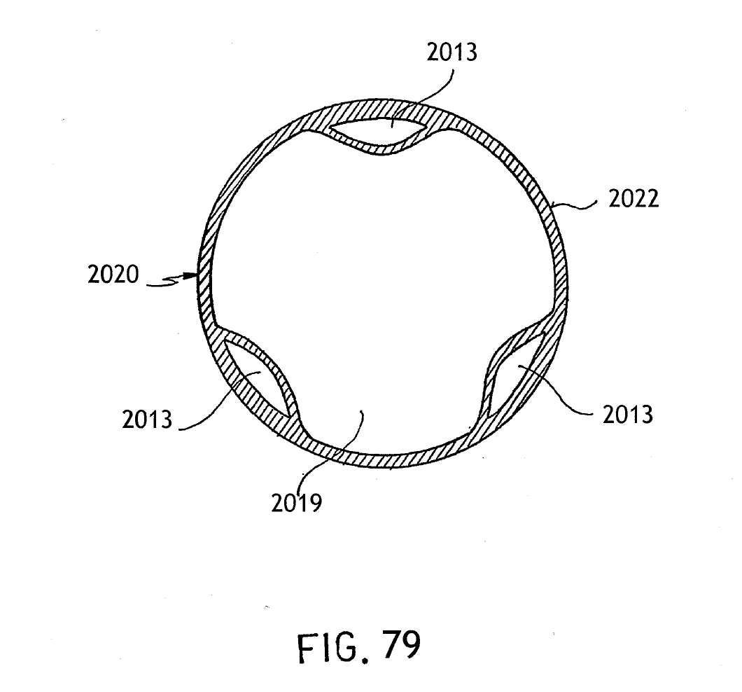

With reference to FIGS. 16-71, the number 1001 indicates as a whole an endolumenal device for delivering and deploying an endolumenal expandable prosthesis or inflatable catheter. Although the embodiment of FIGS. 16-71 generally relate to an asymmetrically expanding expandable device, certain components thereof may be relevant to the embodiment hereinbefore described and such components may be combined with those hereinbefore described. FIGS. 72-79, illustrated another embodiment in connection with the reference number 2001 having a generally symmetrically expandable expansion device, wherein a plurality of guidewire lumen aid in positioning near a vascular bifurcation. These devices are suitable for deploying an expandable endolumenal prosthesis with a bifurcation having one principal conduit and at least one secondary conduit.

Said endolumenal device includes an elongated body 1002 having a distal end portion 1003 and a proximal end portion 1004. For example, said elongated body 1002 is between 100 cm and 160 cm in length, and preferably between 115 cm and 140 cm. The distal end portion 1003 includes expansion means, 1005, which can be removably engaged with an endolumenal expandable prosthesis 1006. Said expansion means 1005 can adapt said prosthesis 1006 from a radially collapsed to a radially expanded position, in a manner which will be described in greater detail below. The expansion means 1005 include a distal portion 1007 of the expansion means a proximal portion 1008 of the expansion means and a central portion 1005a of the expansion means to which the prosthesis 1006 can be attached. In one embodiment, the distal portion of the elongated body 1003 extends beyond the expansion means 1005 in an apical portion 1009. At the proximal end of the proximal end portion 1004 of the elongated body 1002, there are means 1010 for connecting the endolumenal device 1001 to a device of a type known per se for the controlled activation of the expansion means 1005.

The endolumenal device 1001 also includes guidewire tracking means 1011 which extend at least partially along the elongated body 1002. For example, said means 1011 extend along the distal end portion 1003 of the elongated body 1002 close to the expansion means, 1005 (FIG. 16).

Advantageously, the active portion of the expansion means may be longitudinally attached to the elongated body to expand the prosthesis eccentrically to one side with respect to the elongated body so that the other side of the elongated body is left free of said expanded active portion.

Advantageously, said guidewire tracking means 1011 comprise a first guidewire lumen 1012 which extends at least partially inside the elongated body 1002.

In one embodiment of the invention, a first guidewire lumen 1012 and a second guidewire lumen 1013 extend completely inside the elongated body 1002. Distal ports 1014, 1015 and proximal ports 1016, 1017 make said first and second lumens 1012, 1013 able to receive guidewires 1024, 1025 (FIG. 34).

In one embodiment of the invention, the first guidewire lumen 1012 extends entirely inside the elongated body 1002. The first guidewire lumen 1012 may extend between a distal port 1014 and a proximal port 1016, connecting with the walls of the elongated body 1002 only in the vicinity of the respective distal and proximal ports (FIG. 20).

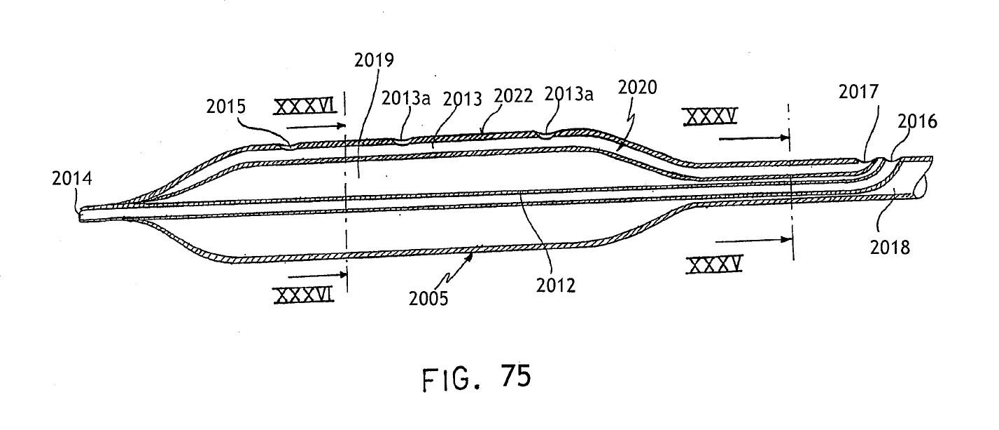

A second guidewire lumen 2013, also extending inside the elongated body 2002, remains attached to the wall of the expansion means 2005, preferably from a distal port 2015 to a proximal port 2017 (FIGS. 75, 76). In one possible embodiment, the second guidewire lumen 2013 remains attached to the expansion means 2005 for a portion of its length, for example comparable to the length of the expansion means 2005, from the distal port 2015. A second portion of the second guidewire lumen 2013 approaching the proximal port 2017 remains inside the device but separate from the wall of the actual body and independent of the first guidewire lumen 2012 (FIGS. 73, 74).

The distal ports 1014, 1015 and proximal ports 1016, 1017 allow guidewires 1024, 1025 to fit into said first and second lumens 1012, 1013 (FIG. 34).

The distal ports 1014, 1015 are preferably spaced out along the elongated body 1002. For example, the distal port 1014 of the first guidewire lumen 1012 is provided at the distal end of the apical portion 1009, and the distal port 1015 of the second guidewire lumen 1013 is provided near the distal end of the expansion means 1005 (FIGS. 16-18, 21a, 21b, 33-38). The proximal ports 1016, 1017 are preferably positioned in the portion of the elongated body 1002 that lies between its proximal end and the expansion means 1005. For example, said ports 1016, 1017 are located at a distance ranging between 90 cm and 130 cm, and preferably between 105 cm and 115 cm, from the proximal end, or from the connector means 1010 (FIG. 16).

In one embodiment of the invention, the second guidewire lumen 2013 has at least one additional port 2013a between the distal port 2015 and the proximal port 2017. For instance, the embodiment seen in FIG. 75 has two additional ports 2013a. One possible embodiment may have a plurality of additional ports 2013a intermediate between the proximal port 2017 and the distal port 2015, preferably arranged so that the distance between two consecutive ports is constant.

According to one embodiment, said endolumenal device is a balloon catheter for angioplasty, 1001. Said balloon catheter 1001 comprises a tubular catheter 1002, a proximal connector 1010, and an inflatable balloon 1005.

The catheter body 1002 is tubular. The proximal portion 1004 of said tubular body 1002 is designed to support and push the distal portion 1003. Therefore said proximal portion 1004 is less flexible than the distal portion, which must be flexible in order to be able to enter the peripheral branches of a vessel. For example, said proximal portion 1004 is made of a biocompatible material, such as biomedical steel or nylon™. Moreover, said proximal portion 1004 is designed to be received in a guide catheter (not shown and known per se) which is necessary for maintaining accessibility of the lumen of the vessel on which it is necessary to operate even when the endolumenal device 1001 is withdrawn. Said guide catheter is also necessary for introducing, for example, a radio-opaque contrast medium into the vessel. The proximal portion 1004 of the catheter body, 1002 includes an inflation lumen, 1018 (FIGS. 18, 19 and 19a, 21b). Said lumen 1018 extends from the proximal end of the catheter body 1002 to the inflatable balloon 1005.

The proximal connector, 1010, for example a connector commonly known as a “Luer”, is provided at the proximal end of said portion 1004 and forms the abovementioned means of connection of the endolumenal device 1001 to the device for the controlled activation of the balloon 1005. For example, said connector connects the inflation lumen 1018 of the balloon 1005 to a pressurized fluid source.

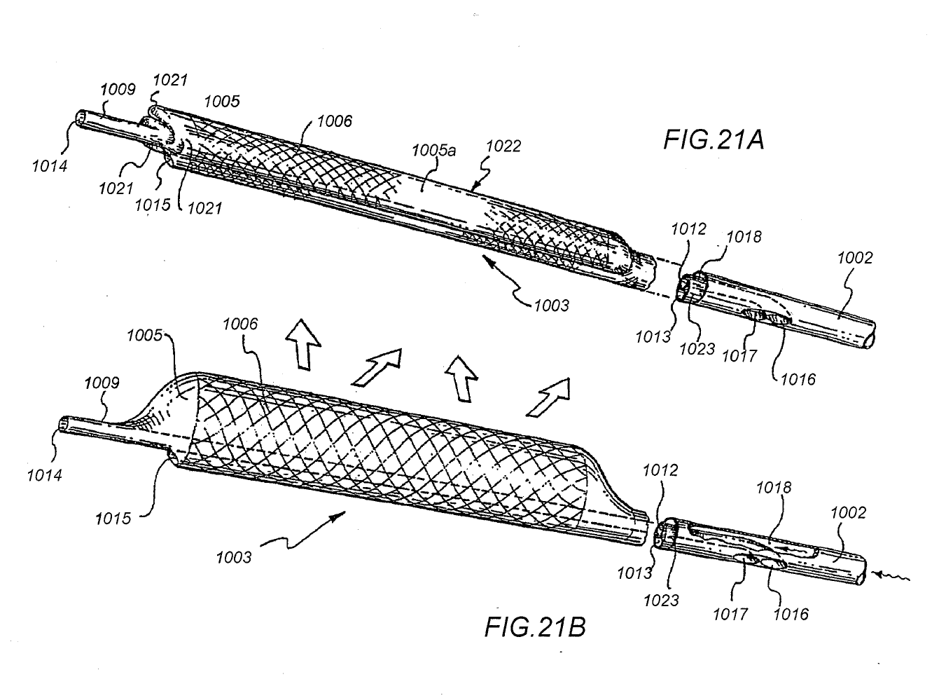

The balloon 1005 is associated with the distal portion 1003 of the catheter body 1002 to form an inflation chamber 1019 which at least partially surrounds the catheter body (FIG. 18). The inflation chamber 1019 is delimited by a balloon wall 1020 equipped with an external envelope 1022. Said inflation chamber 1019 is in communication with the inflation lumen 1018. In one embodiment, the balloon includes, between a distal portion 1007 and a proximal portion 1008, a central portion 1005a. Said central portion 1005a, when it is in a radially expanded, or inflated position is roughly cylindrical. The balloon wall 1020 in one embodiment is non-extendable or rigid when subjected to pressurized fluid. Therefore the balloon wall 1020, when it is in a radially collapsed position, is folded around the catheter body 1002, for example it is threefolded or, in other words, forms three folds 1021 (FIG. 21a). By means of the external envelope 1022, the balloon wall 1020 can be removably fitted with an endolumenal prosthesis. For example, the external envelope is removably fitted with an endovascular stent of metallic tubular mesh, plastically deformable from a radially collapsed condition to a radially expanded condition, which can be fixed by pressure to the internal surface of a vessel wall. For this reason, the diameter of the central cylindrical portion 1005a, when the balloon is radially expanded or inflated by pressurized fluid injected through the inflation lumen 1018, is such as to fix said prosthesis to the wall of the vessel by pressure (FIG. 21b).

In one embodiment of the invention, a longitudinal portion of the balloon wall 1020 is associated internally with the catheter body 1002. In other words, said wall 1020 is fixed along its entire length to the catheter body, so that when the balloon 1005 changes from the radially collapsed or deflated position to the radially expanded or inflated position, said balloon 1005 will extend preferably eccentrically or asymmetrically with respect to the catheter body 1002, or in other words, on only one side of the body (FIGS. 18, 20 and 21b).

The distal portion 1007 and the proximal portion 1008 of the balloon 1005 are advantageously pointed in shape. In particular, said portions are frustoconical.

Advantageously, the tubular catheter body 1002 includes sheath means or sleeve means 1023, for example a flexible conduit. For example, said sheath means are an integral part of the elongated body. The sheath means 1023 include a tubular body through which run a number of longitudinal lumens, 1012, 1013 forming the abovementioned guidewire lumens. The guidewire lumens 1013, 1014, or sections of these, advantageously run in parallel along the elongated body. Said lumens debouch at the ends of the sheath means with the abovementioned guidewire ports 1014, 1015, 1016, 1017. Said sheath means 1023 are located inside the tubular catheter body 1002 in such a way as to leave a space (which forms the abovementioned inflation lumen 1018) along the entire length of that portion of the catheter body 1002 which is situated between the proximal connector 1010 and the balloon 1005. Preferably, said sheath means are attached for their entire length to the portion of the wall of the catheter body opposite the inflation chamber 1019 (FIGS. 18, 19, 19a, and 21b). The ends of the sheath means 1023 are attached to the wall of the catheter body in such a way as to make the guidewire lumens accessible from outside the catheter body through the guidewire ports.

It is particularly advantageous when said sheath means 1023 are attached to the catheter body so that they debouch in a first distal guidewire port 1014 of the first guidewire lumen 1012, distant from a second distal guidewire port 1015 of the second guidewire lumen 1013.

In particular, said sheath means extend to the tip of the distal portion 1003 of the catheter body 1002 in such a way as to debouch with the first distal guidewire port to the tip of the apical tract 1009.

In one possible embodiment (FIGS. 73 and 75), the sheath means 2023 comprise a tubular body pierced longitudinally by the first lumen 2012 forming the guidewire lumen described above. This sheath extends through the elongated body 2002 from the proximal end portion 2004 to the distal end portion 2003. The second guidewire lumen 2013, or at least a section of the latter, advantageously runs through the elongated body 2002 attached to the expansion means 2005, that is in the balloon wall 2020. The wall part 2020 that houses the second guidewire lumen 2013 is preferably the balloon part that can expand asymmetrically with respect to the elongated body 2002. In one possible embodiment, the guidewire lumen 2013 runs inside the balloon wall 2020 from its proximal port 2017 to its distal port 2015 (FIG. 75). In a different embodiment, only a portion of the second guidewire lumen 2013 runs inside the balloon wall 2020, preferably the portion corresponding to the extension of the expansion means 2005, that is extending from the distal port 2015 to an intermediate point of the wall. From this intermediate point and as far as the proximal port 2017, the second lumen 2013 may run independently inside the elongated body 2002 defined by other sheath means 2023a (FIG. 73).

The first lumen 2012 has an open end at one end of the sheath means with the above-described guidewire ports 2014 and 2016. The lumen 2013 ends at the end of the balloon wall 2020 with the above-described guidewire ports 2015 and 2017 and, if desired, with the additional one or more ports 2013a. Said sheath means 2023, and, if desired, the additional sheath means 2023a, are located inside the tubular catheter body 2002 in such a way as to leave a space, forming the abovementioned inflation lumen 2018, along the full length of the catheter body 2002 situated between the proximal connector 2010 and the balloon 2005.

In a further embodiment of the invention, for example thanks to the asymmetrical position of the balloon 1005 with respect to the catheter body 1002, the second distal guidewire port 1015 is positioned along the catheter body 1002 so as to allow the second guidewire lumen 1013 to debouch at the distal end of the central portion 1005a of the balloon, or in other words, so as to be positioned just outside the prosthesis 1006 attachable to the balloon 1005 (FIGS. 16 to 21b).

In a further embodiment of the invention, the second distal guidewire port 1015 is positioned along the catheter body in such a way that the second guidewire lumen 1013 debouches at a point located between the distal portion 1007 and the proximal portion 1008 of the balloon 1005, and in particular at a point of wall opposite the central portion 1005a attachable to the prosthesis 1006. For example, said port 1015 is located near the centre line of said central portion, 1005a. Preferably, the prosthesis 1006, which can be attached to said catheter 1001, has a window 1026 designed to prevent obstruction of said distal guidewire port 1015 when it is fitted on the balloon 1005. For example, the prosthesis 1006 has a wider mesh 1026 than the other meshes of the prosthesis, and at the same time of a size close to that of the ostium of access to the lumen of the branch on which it is necessary to operate, or only slightly smaller. Alternatively, the balloon can be fitted with a number of prostheses, placed side by side in order to avoid obstructing said port 1015.

Preferably, the proximal guidewire ports 1016, 1017 are located in a portion of the catheter body 1002 which, during use of the catheter 1001, remains sheathed in the guide catheter. Alternatively, said ports 1016, 1017 are located at the proximal end of the catheter body. In this case the balloon catheter 1001 is fitted with a proximal connector 1010 with at least two channels. A first channel for the admission of the pressurized fluid into the inflation lumen 1018, and a second channel for passing the guidewires 1024, 1025 along. For example, said proximal guidewire ports are located at a distance from the tip of the catheter ranging between 15 cm and 35 cm, and preferably between 20 cm and 30 cm.

Advantageously, radio-opaque markers 1030 and 1031 are associated with the catheter body 1002 (FIG. 18). For example, said markers are located along the catheter body 1002 at the distal and proximal ends of the prosthesis 1006.

Said catheter body also includes radio-opaque markers for the identification of the position along said body of the distal 1014, 1015, and/or proximal 1016, 1017 guidewire ports of the guidewire lumens 1013, 1014. In another embodiment, both the guidewire lumens 1012 and 1013, or at least a section of these, advantageously run in the wall of the elongated body 1002. This section preferably corresponds to the longitudinal section of the expansion means 1005.

In another embodiment, there are more than two guidewire lumens in the balloon wall 2020, preferably three arranged at 120° from each other (FIG. 79). In this latter embodiment, it is possible to avoid the guidewire lumen 1012 positioned inside the elongated body 1002, as described earlier.

### A. Kits Including an Endolumenal Device and One or More Guidewires

The subject of the present invention also comprises a kit for delivering and positioning an endolumenal expandable prosthesis. Although described hereinblow in connection with the devices 1001, 2001, these disclosure are applicable to the apparatuses of FIGS. 1-15 as well.

The kit comprises an endolumenal device 1001, as described above, at least one pair of guidewires 1024, 1025, and at least one expandable prosthesis 1006 radially associated with the expansion means 1005 of said endolumenal device 1001. Said prosthesis comprises a tubular prosthesis body adaptable from a radially collapsed condition, of minimal external diameter, to a radially expanded condition, of extended external diameter greater than the collapsed external diameter.

For example, said kit for delivering and positioning an endolumenal expandable prosthesis comprises at least one first radially expandable prosthesis associated with the proximal portion of the expansion means of said endolumenal device and also comprises at least one second radially expandable prosthesis associated with the distal portion of the expansion means of said endolumenal device, or alternatively a single prosthesis overlapping said proximal and distal portions of the expansion means.

Each of the guidewires of said kit includes means of identification, such as for example the color of at least a proximal portion of the guidewire, or a diameter of the cross section of a proximal portion of the guidewire which differs for each guidewire.

Said guidewires advantageously comprise an elastically flexible distal end portion.

In particular, said guidewires include initial proximal sections which are positionable along a proximal section of path common to all the guidewires, and secondary distal sections which are positionable along distal sections of path which diverge and form with said proximal section of path a bifurcation. It is particularly advantageous for at least one of said guidewires to include an elastically flexible distal portion, which extends at least to straddle said bifurcation.

It is furthermore advantageous for said guidewires to include radio-opaque markers, for example located at the tip of the distal portion.

### B. Further Methods for Treating Patients

A description of the working of endolumenal devices according to this invention follows. Although discussed mainly with reference to FIGS. 16-79, these disclosure can be applied to the embodiments of FIGS. 1-15.

In particular, the operations necessary for guiding an endolumenal device along guidewires 1024, 1025 are described below. Said guidewires are located along a common proximal section of path and a diverging distal section of path, forming a bifurcation between said sections. The above method comprises the following stages:
said endolumenal device is fitted onto a proximal end of a first guidewire so that said first guidewire is received in a first guidewire lumen through its distal guidewire port;
said endolumenal device is fitted onto a proximal end of a second guidewire so that said second guidewire is received in a second guidewire lumen through its distal guidewire port;
said endolumenal device is advanced along said guidewires until at least part of the distal end portion of the elongated body is positioned beyond the bifurcation of the guidewires.

Advantageously, it is possible to envisage a further method of guiding an endolumenal device along guidewires 1024, 1025, in which said guidewires are positioned along a common proximal section of path and a diverging, distal section of path, forming between said sections a bifurcation. This further method includes the following stages:
said endolumenal device is fitted onto a proximal end of a first guidewire so that said first guidewire is received in a first guidewire lumen through its distal guidewire port;
said endolumenal device is fitted onto a proximal end of a second guidewire so that said second guidewire is received in a second guidewire lumen through its distal guidewire port;
said endolumenal device is advanced along said guidewires until at least part of the distal end portion of the elongated body lies on a distal divergent section of path of one of the guidewires.

The steps of a method for fitting radially expandable prostheses to the walls of branches forming a ‘T bifurcation’ 1032 are described below (FIGS. 27 to 32e). Said bifurcation 1032 comprises a principal conduit 1033 and a secondary conduit 1034 that branches off from a wall of the principal conduit 1033. The abovementioned method comprises the following steps:

A kit as described above, and in particular a kit which comprises an endolumenal device having a distal guidewire port located inside a central portion of the expansion means, is prepared.

Then, through a proximal section of the principal conduit, a first guidewire is positioned in the principal conduit so that it passes the bifurcation, and a second guidewire is positioned in the secondary conduit. Said guidewires are positioned in such a way as to follow an initial proximal section of path together and second distal sections of path that diverge at said bifurcation (FIG. 27).

Next, a first endolumenal device equipped with a radially expandable prosthesis, is fitted onto a distal end of the second guidewire, so that said second guidewire is received in a guidewire lumen of the endolumenal device through a distal guidewire port located on the tip of its elongated body.

Said first endolumenal device is inserted into said conduits following the proximal and then the distal sections of path of the second guidewire in order to position the radially expandable prosthesis in the secondary conduit so that its proximal edge is positioned near a mouth of said secondary conduit (FIG. 28).

Said expandable means are then activated so that said prosthesis is in its radially expanded condition and fixed by pressure to the wall of the secondary conduit (FIG. 29).

Next, said expansion means are withdrawn and the first endolumenal device is withdrawn from the second guidewire until it has been removed from the conduits.

A second endolumenal device equipped with a radially expandable prosthesis is fitted onto a proximal end of the first guidewire so that said first guidewire is received in a first guidewire lumen through its distal guidewire port located on the tip of the endolumenal device and said second endolumenal device is fitted onto a proximal end of the second guidewire so that said second guidewire is received in a second guidewire lumen through its distal guidewire port located on the portion of elongated body that lies between a distal and a proximal end of the expansion means.

Said endolumenal device is inserted into the principal conduit and slid along the proximal section of path of the guidewires until a distal portion of the endolumenal device, located between the tip of said device and the distal guidewire port of the second guidewire lumen, is positioned beyond the bifurcation (FIG. 31).

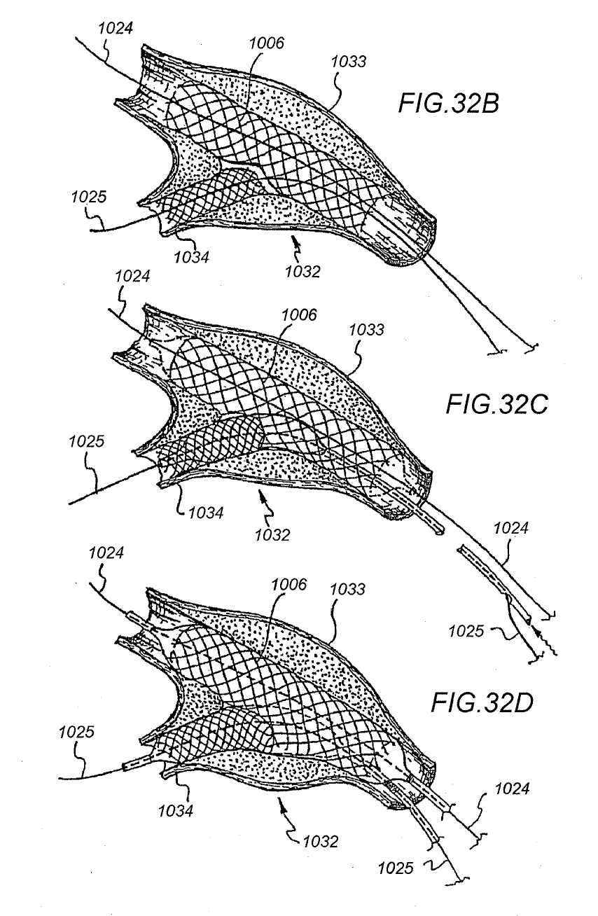

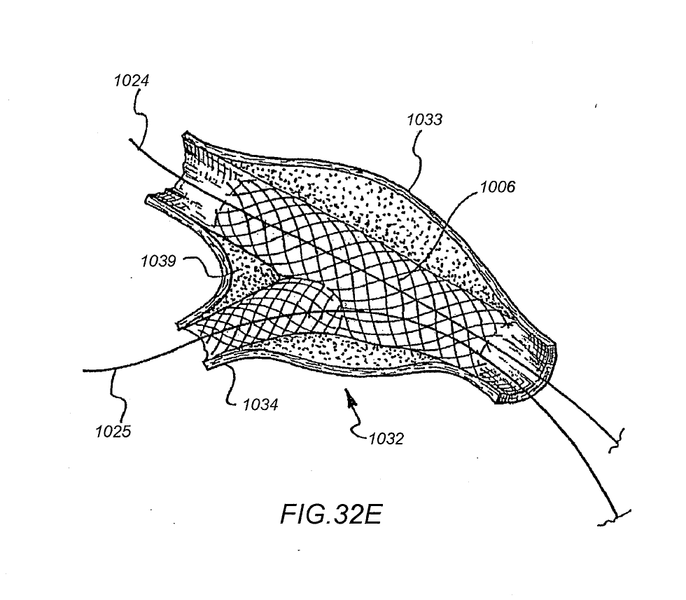

The expandable means of said second device are activated so as to bring said prosthesis into its radially expanded condition and fixed by pressure to the wall of the primary conduit and straddling the bifurcation (FIG. 32a).

Finally said expansion means are withdrawn and then the second endolumenal device is withdrawn from the guidewires until it has been removed from the conduits.

Further steps which make it possible to adapt the previously implanted prostheses in order to cover the lesion completely are described below.

A third endolumenal device without a prosthesis is fitted onto the second guidewire, positioning it to straddle the bifurcation so that a distal portion of the expansion means enters the secondary conduit and a proximal portion of the expansion means is positioned in the principal conduit.

The expansion means of the third device are then activated so as to adapt a portion of the prosthesis in the principal conduit facing the mouth or lateral window of the secondary conduit to the shape of the lumen of said secondary conduit (FIG. 32c).

Said expansion means are withdrawn and then the third endolumenal device is withdrawn from the second guidewire until it has been removed from the conduits.

By inflating the third endolumenal device (for example a balloon catheter for angioplasty) straddling the bifurcation, the mesh of the prosthesis implanted in the principal conduit is molded so that it surrounds the ostium of the secondary conduit perfectly, and guarantees perfect coverage of the damaged area (FIG. 32c). Alternatively, particularly in the case of larger diameter or larger bore conduits it is possible to insert two balloon catheters simultaneously, fitting them on the guidewires 1024, 1025, so that they are paired and straddle the bifurcation, one in the principal conduit and the second partially in the secondary conduit and partially in the principal conduit. Simultaneous expansion of the two balloons both shapes the prostheses so that they match and form a continuous support structure which covers the entire extension of the lesion and creates, in the area of the bifurcation, a funnel-shaped area which joins the principal and the secondary branches and promotes non-vortical circulation of fluid in the conduits or vessels.

The stages of the method described above may also be reversed, implanting first the principal vessel and then the secondary vessel.

In view of the above procedures it is evident that the implanting of a prosthesis in the principal vessel causes the plaque 1039 to obstruct the ostium of the secondary vessel or vice versa. Thanks to the fact that, using the device according to the invention, the application of a first prosthesis in a vessel is always carried out leaving a second guidewire in a second branch, in spite of the presence in the mouth of the same of a wall of plaque caused by “snow-plow” or “plaque-shifting”. It is therefore always possible to insert in the second branch a device for the application of a second prosthesis. Using known prior-art devices it is not possible to operate simultaneously with two guidewires always present in the two branches of the bifurcation, because a second guidewire not positioned inside the prior-art device would be externally walled by the prosthesis and rendered unusable. In other words, with the prior-art device it is necessary to proceed using only one guidewire. With the device according to the invention, however, it is possible to effect the swift exchange of the endolumenal device on guidewires which remain in situ, it being possible to withdraw the endolumenal device from a first branch of the bifurcation to reinsert the same device or a second device in a second branch with extreme rapidity.

The steps for a further method for fitting radially expandable prostheses to the walls of the branches of conduits forming a ‘Y bifurcation’ 1035 are described below. Said bifurcation comprises a proximal principal conduit 1036 and a first and a second secondary distal conduits 1037, 1038 which branch off from a distal end of the principal conduit, forming between them a carina. Said method comprises the following steps.

A kit as described above is prepared, and in particular a kit comprising an endolumenal device fitted with a distal guidewire port located near the distal edge of a prosthesis fitted on the expansion means, and a second distal guidewire port located at the tip of the device, or apical port.

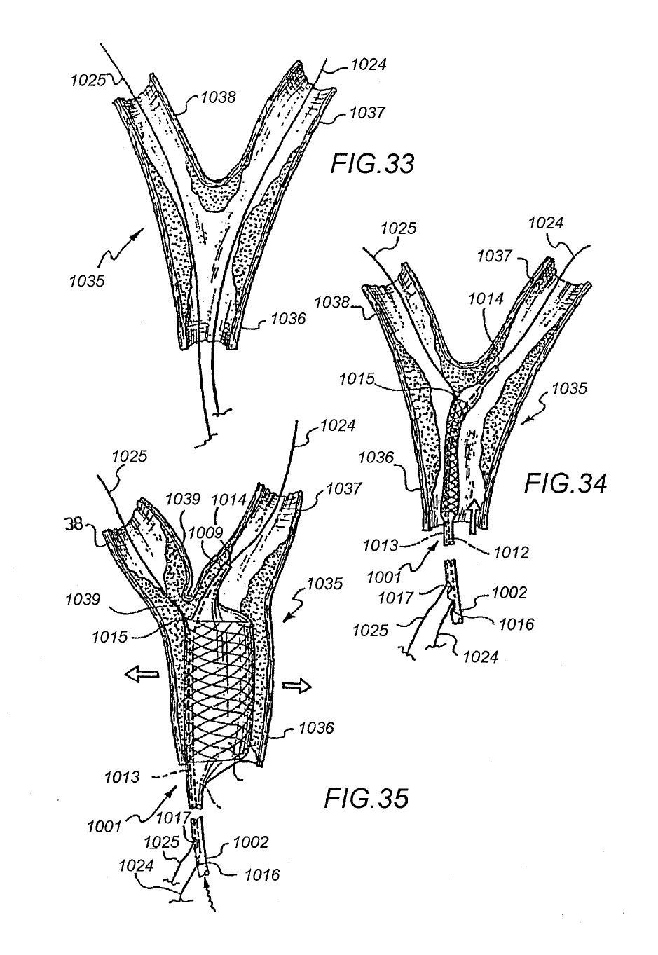

Through the principal conduit a first guidewire is positioned in the first secondary distal conduit and a second guidewire in the second secondary distal conduit, said guidewires being positioned so as to follow a first proximal section of path together and second distal section of path that diverge after said bifurcation (FIG. 33).

A first endolumenal device equipped with a radially expandable prosthesis is fitted onto a proximal end of the first guidewire, so that said first guidewire is received in a guidewire lumen of the endolumenal device through its distal guidewire port located at the tip of its elongated body.

Said first endolumenal device is fitted onto a proximal end of the second guidewire so that said second guidewire is received in a second guidewire lumen through its distal guidewire port located near the stent distal edge, just beyond the prosthesis.

Said first endolumenal device is inserted into said conduits following the proximal section of path until the carina is positioned against the elongated body and near the distal guidewire port positioned near the distal end of the expansion means (FIG. 34).

Said expandable means are activated so as to bring said prosthesis into its radially expanded condition, fixed by pressure to the wall of the principal conduit (FIG. 35).

Said expansion means are withdrawn and the first endolumenal device is then withdrawn from the guidewires.

A second endolumenal device equipped with a radially expandable prosthesis is fitted onto a proximal end of the first guidewire so that said guidewire is received in a guidewire lumen through its distal guidewire port located on the tip of said second endolumenal device.

A third endolumenal device equipped with a radially expandable prosthesis is fitted, at the same time as the second endolumenal device, onto a proximal end of the second guidewire so that said second guidewire is received in a guidewire lumen through its distal guidewire port located on the tip of said third endolumenal device.

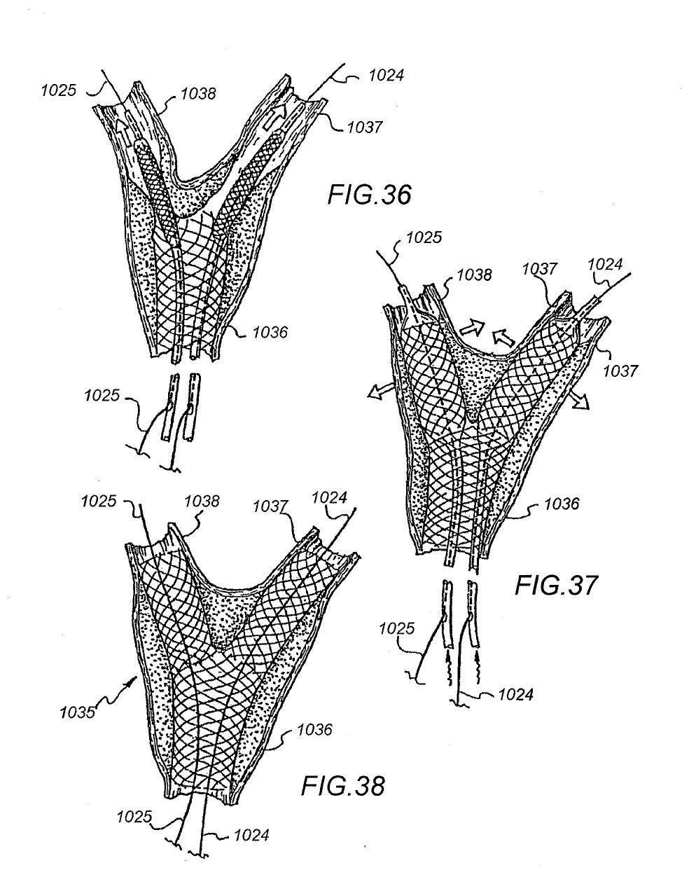

Said second and third endolumenal devices are simultaneously inserted into the principal conduit and slid along the proximal section of path of the guidewires and then along the respective distal sections of path of said guidewires, until the expansion means are positioned in a proximal portion of said first and second secondary conduits, so that a proximal edge of the expansion means is positioned near the carina. In particular, care is taken to ensure that the proximal edge of both the second and third prostheses is in contact with the distal edge of the first prosthesis, already positioned and expanded in the principal lumen (FIG. 36).

The expansion means of said second and third endolumenal devices are activated in order to bring the respective prostheses into a radially expanded condition fixed by pressure to the walls of said first and second conduits (FIG. 37).

Said expansion means are withdrawn and then the second and third endolumenal devices are withdrawn from the guidewires until they have been removed from the conduits (FIG. 38).

The above description shows how the use of at least two guidewire lumens which extend at least partially along the inside of the elongated body makes it possible to fit the endolumenal device simultaneously on at least two guidewires. In this manner, once at least two guidewires have been inserted in the branches of a bifurcation, it will be possible to insert and withdraw the endolumenal device from a first branch of the bifurcation without ever losing rapid access to all the branches already negotiated, i.e. reached by guidewires. In other words, it will be possible to maintain uninterrupted access or vascular approach to all the branches of the vascular system on which it is necessary to operate and in which a guidewire has been inserted or, in yet other words, using the device proposed it is no longer necessary to break through the wall of plaque 1039 which obstructs the ostium of the branch by “snow-plow” or “plaque-shifting.”

Thanks to the endolumenal devices according to the invention it will also be possible to position accurately a first endovascular prosthesis in the principal vessel always with precise positioning and complete distension or application of the prosthesis over the entire area of the lesion, thus reducing the probability of re-stenosis and avoiding the pitfalls of the known techniques.

Advantageously, the endolumenal device proposed allows extreme flexibility and modularity in the application of the endolumenal prostheses. Thus, if the expansion means are positioned exactly straddling the bifurcation it is possible to implant endolumenal prostheses of exactly the correct length and diameter for the dimensions of the segment of damaged vessel to be treated, by means of the proximal and distal portions of the expansion means.

With further advantage, each portion of the expansion means makes it possible to implant a number of endolumenal prostheses of optimal diameter and length for the anatomy of the damaged vascular branch.

When expansion means fitted to the endolumenal prostheses are in the collapsed position, the device according to the invention is of reduced transverse bulk, making it possible to reach peripheral branches extremely easily and rapidly (trackability).

Together with the versatility of application of prostheses adapted to different branches of the bifurcation, the device proposed also makes it possible to join prostheses, or, in other words, it allows total coverage of the damaged area, avoiding prolapse of atheromatous material and reducing the probability of re-stenosis.

A further advantage derives from the fact that, using the endolumenal device according to the invention, the geometry of the prosthesis is not distorted and the vascular anatomy is respected. In contrast, distortion of the prosthesis is inevitable when endolumenal devices according to the prior art are used.

Obviously, variations and/or additions to what is described above and illustrated may be envisaged.

Alternatively to a balloon with rigid walls threefolded onto the catheter body for insertion into the lumen of a vessel, as described above, it is possible to envisage the use of a compliant or extensible balloon.

### III. Additional Variations

The catheters of the types described above, “single-operator exchange” or “monorail”, may alternatively be of the “over-the-wire” type, that is with opening of the proximal guidewire lumens at the proximal end of the elongated body.

One of the at least two guidewire lumens may always be occupied by a guidewire and may be inserted in the conduit, or vessel, together with the endolumenal device. Preferably, in this case the guidewire is fastened to or is an integral part of the elongated body of the endolumenal device, for example extending from the apical portion of this (“fixed-wire”).

The catheter may also be of the perfusion balloon type in which passages are provided for fluid flow when the balloon is inflated: these provide communication between the portions of elongated body above and below the expansion means (passages for the blood in the body to prevent temporary occlusion of the vessel during the application of the prosthesis and the inflation of the balloon).

The endovascular prosthesis may be modular. For example it is possible to provide a series of prostheses of set diameters and a series of set lengths which the operator can attach to the proximal and distal portions of the expansion means, making them extremely flexible or, in other words, making it possible to adapt the prosthesis perfectly to the pathological requirements of the moment, or in other words, to the size of the lesion and the bore of the lumen of the vessel on which it is necessary to operate.

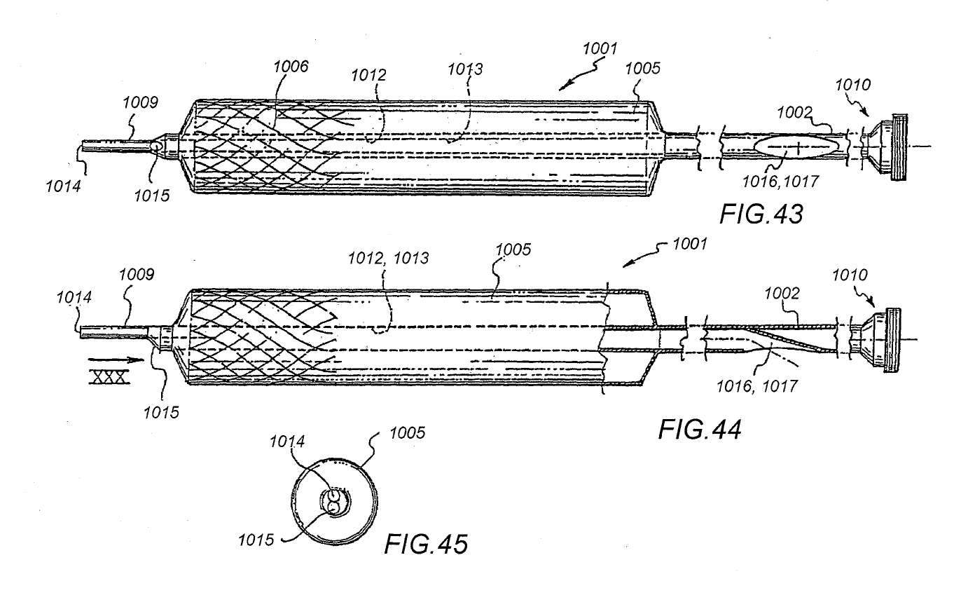

As an alternative to the above description, illustrated by FIGS. 18 and 23, at least one portion of said at least one pair of guidewire lumens 1012, 1013 forms a single guidewire lumen (FIGS. 43, 44, and 45).

Advantageously, the distal guidewire port 2015 of a second guidewire lumen 2013 is located near a proximal end of the expansion means 2005 (FIGS. 72, 73). A conduit 1027, for example a flexible conduit, preferably longitudinally elastic, is functionally connected through its proximal port 1028 to said distal guidewire port 1015. Said conduit 1027 has a distal guidewire port 1015a and, being attached along its entire length to the external surface of the expansion means 1005, said distal port 1015a is located in the central portion 1005a of said expansion means 1005. In this case, advantageously, the expansion means, for example a balloon 1005, expand symmetrically to the elongated body 1002.

In a further embodiment of the invention, said expansion means are designed to hold a self-expanding prosthesis in a radially folded position and release it in a controlled manner so that it takes up a radially expanded position. Said expansion means include a sheath designed to receive in a sheath lumen said self-expanding prosthesis. Said sheath can advantageously be adapted in controlled manner from a first constricted position in which the self-expanding prosthesis is confined in said lumen of the sheath, to a second released position, in which said prosthesis is released from said lumen of the sheath so that said prosthesis is radially free, to bring itself into the radially expanded condition.

Such a device can be advantageously used in the artificial conduits of biomedical equipment that connects up to the patient's body. For example, a device of the type described above can be used for delivering, positioning and installing an element for the repair of the walls of a conduit accidentally damaged during the use of the abovementioned machinery.

Advantageously, the endolumenal device 1001 comprises at least a guidewire lumen 1012 or 1013 extended completely inside the elongated body 1002.

With further advantage, the active portion of the expansion means is entirely associated to the elongated body in order to expand said prosthesis exclusively from one side with respect to the elongated body, and in order to leave free from said expanded active portion the other side of the elongated body.

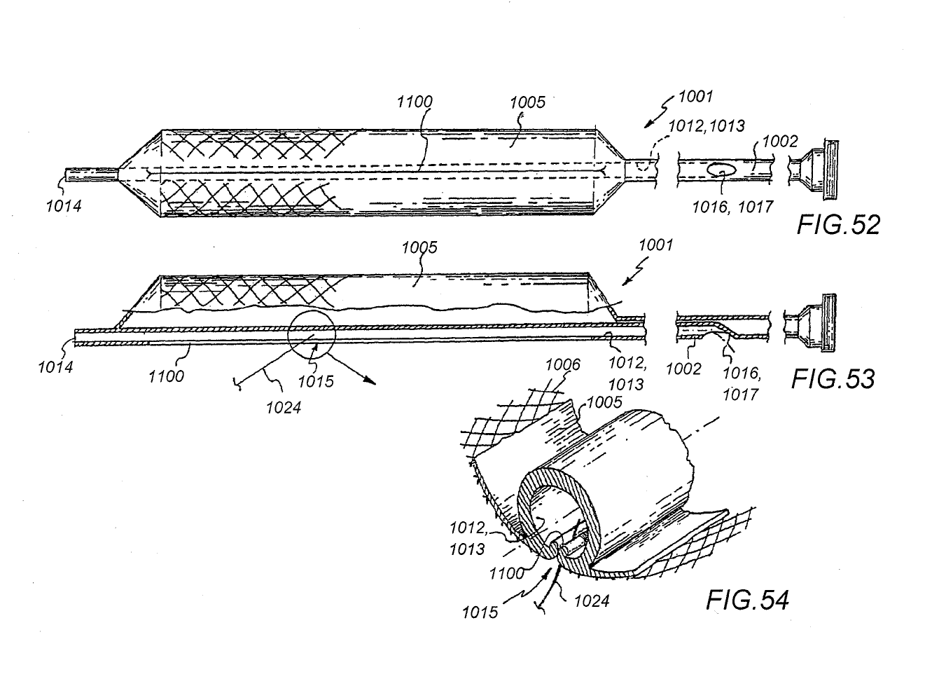

According to one embodiment, the side of the elongated body portion associated to the expansion means and free from said expanded active portion, or free side, is provided with a fissure 1100 suitable for realizing a distal guidewire port 1015 of the at least a guidewire lumen 1012, 1013. It is furthermore advantageous for said fissure 1100 to be extended between the distal end and the proximal end of the elongated body portion associated to the expansion means 1005 (FIGS. 52, 53 and 54).

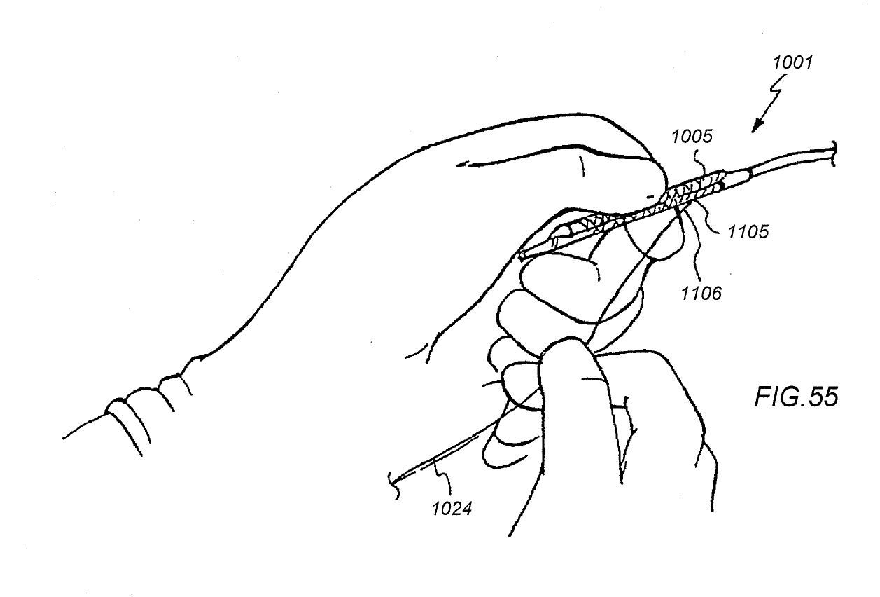

Preferably, the side of the elongated body associated to the expansion means 1005 and free from the expanded expansion means comprises a wall 1105 that partially binds said at least a guidewire lumen 1012, 1013. Said wall 1105 is suitable for being bored by a guidewire end 1106, for example the proximal end, in order to slip said guidewire 1024 through the bored portion of the wall 1105 (FIG. 55).

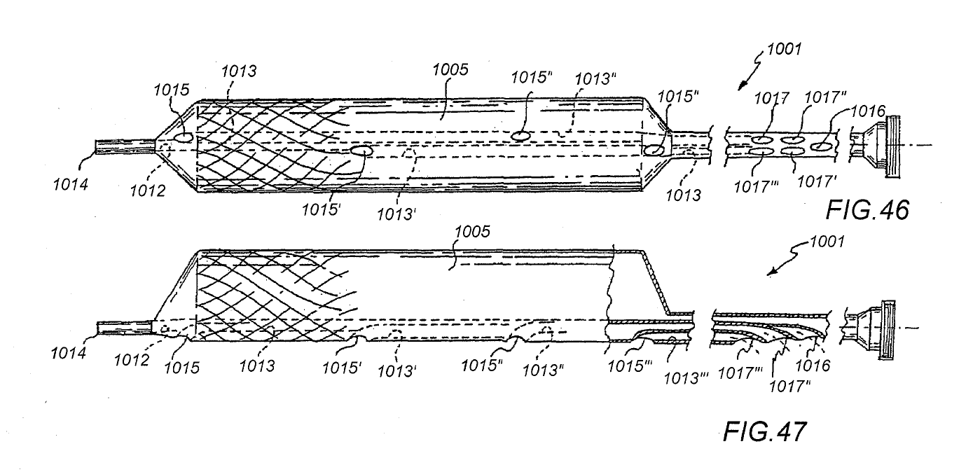

According to a further embodiment, the at least a guidewire lumen 1012 and/or 1013 of the tracking means has a plurality of distal guidewire ports 1014, 1015, 1015^{I}, 1015^{II}, 1015^{III}, 1015^{IV} and/or 1015^{V}, 1015^{VI}, 1015^{VII}, 1015^{VIII}, 1015^{IX}, 1015^{X}, 1015^{XI}, 1015^{XII},1015^{XIII}, 1015^{XIV}, spaced out along said elongated body 1002 (FIGS. 46, 47, 48, 49, 50 and 51).

Preferably, the guidewire tracking means 1011 comprises a plurality of guidewire lumens 1012, 1013, 1013^{I}, 1013^{II}, 1013^{III} associated to each of said distal guidewire ports 1014, 1015, 1015^{I}, 1015^{II}, 1015^{III}, 1015^{IV} (FIGS. 46, 47).

Advantageously, the at least a guidewire lumen 1012 and/or 1013 has a distal guidewire port 1014, or apical port, at the tip of said distal end portion 1003 of the elongated body 1002 (FIGS. 46, 47, 48, 49, 50 and 51).

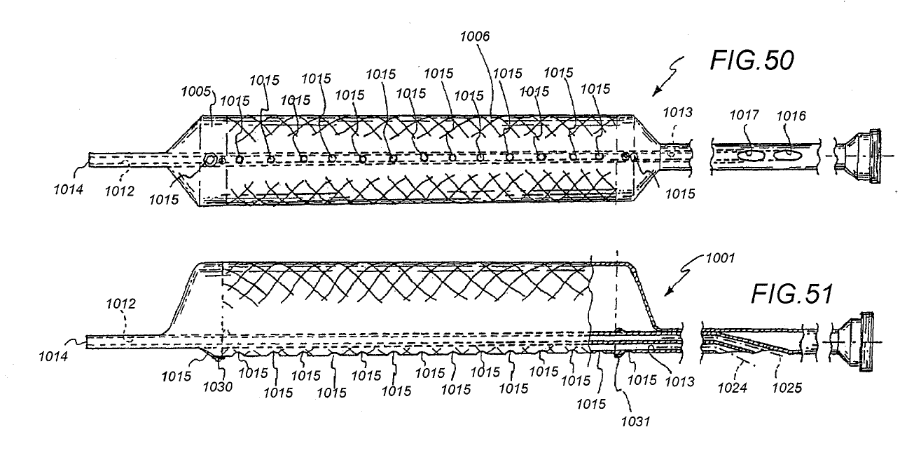

With further advantage, a first guidewire lumen 1012 associated to said apical port 1014 is provided in the body and a second guidewire lumen 1013 is associated to a plurality of distal guidewire ports 1015, 1015^{I}, 1015^{II}, 1015^{III}, 1015^{IV} and/or 1015^{V}, 1015^{VI}, 1015^{VII}, 1015^{VIII}, 1015^{IX}, 1015^{X}, 1015^{XI}, 1015^{XII}, 1015^{XIII}, 1015^{XIV}, or side ports, provided on a side of the elongated body opposite the expansion means (FIGS. 50 and 51).

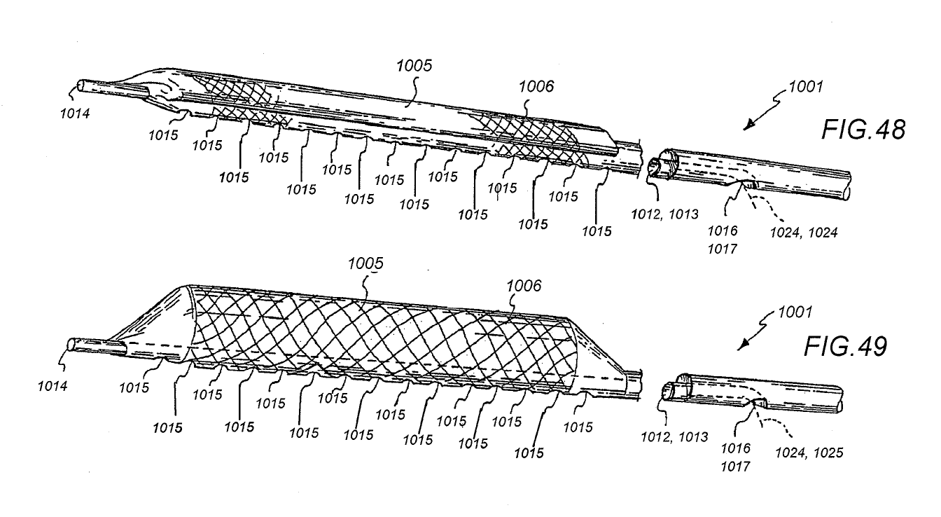

As an extremely advantageous alternative, a single guidewire lumen 1012, 1013 associated to said apical port 1014 is provided in the body and is also associated to a plurality of distal guidewire ports 1015, 1015^{I}, 1015^{II}, 1015^{III}, 1015^{IV} and/or 1015^{V}, 1015^{VI}, 1015^{VII}, 1015^{VIII}, 1015^{IX}, 1015^{X}, 1015^{XI}, 1015^{XII}, 1015^{XIII}, 1015^{XIV}, or side ports, provided on a side of the elongated body opposed to the expansion means (FIGS. 48 and 49).

In a further variation of the invention, the at least a guidewire lumen 1013 has a distal guidewire port 1001 near a distal end of the expansion means 1005.

Advantageously, the at least a guidewire lumen 1013 has at least a distal port 1015, 1015^{I}, 1015^{II}, 1015^{III}, 1015^{IV} and/or 1015^{V}, 1015^{VI}, 1015^{VII}, 1015^{VIII}, 1015^{IX}, 1015^{X}, 1015^{XI}, 1015^{XII}, 1015^{XIII}, 1015^{XIV} in a portion of the elongated body 1002 that lies between a distal end and a proximal end of the expansion means 1005.

### IV. Methods Involving Embolization Containment Devices

In a further variation of the invention, the endolumenal device can be advantageously used in order to improve maneuverability and clinical efficacy of some embolization containment devices (ECD) during coronary angioplasty and stenting.

Actually, a frequent complication of these procedures is the so called “no-flow phenomenon”, consisting of impairment of the blood to flow down to the distal vessels, even though the obstruction has been removed.

This calamitous event is mainly caused by the distal embolization of the thrombus debris, and arterial spasms induced by some vaso-constrictive substances released into the blood stream because of the plaque crumbling and compression during balloon inflation.

These events are frequent when treating recent coronary occlusions in acute myocardial infarction, or when treating coronary lesions with angiographic evidence of a thrombus within the lumen, as in unstable angina.

Therefore, in addition to bifurcated lesion treatment, the proposed device will find large scale application in the situations as described here following.

Most ECD currently in use take the form of an occluding balloon 1102 (FIG. 57b), or of a basket-shaped or an umbrella-shaped device 1101 (FIG. 57c), which necessarily blocks the flow distally of debris, and substances which can cause vasospasms.

An example of such application is described with the following steps:

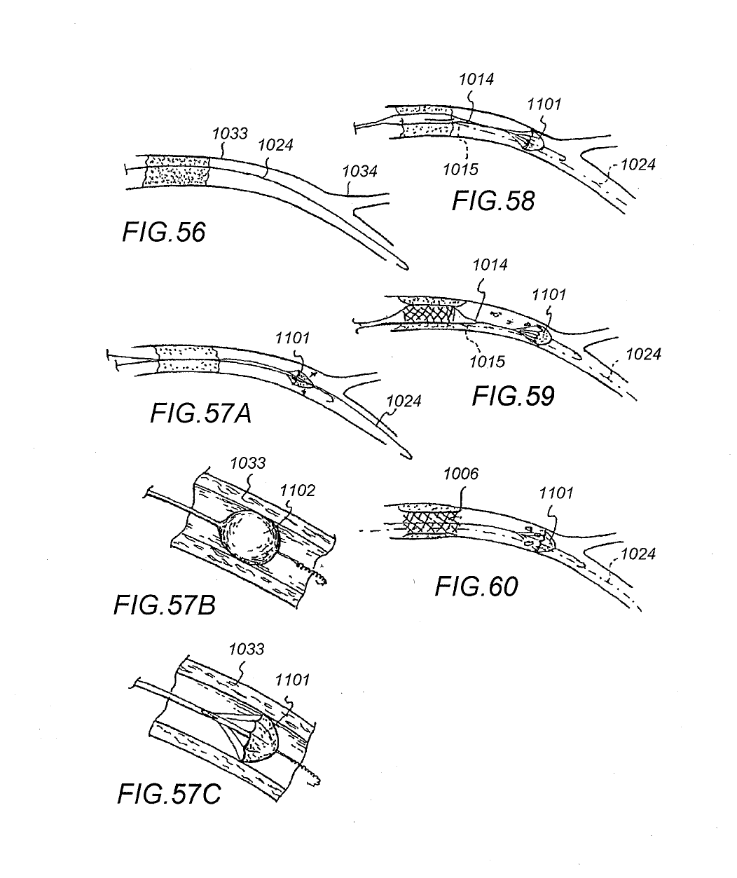

Step 1—a conventional guidewire (cGW) 1024 is advanced beyond the occlusion as a “trailblazer” for the ECD 1101. In fact, these devices have less maneuverability and are more fragile than cGW 1024 and, therefore, can't be used to bore, and to cross an occlusive thrombus (FIG. 56).

Step 2—the ECD 1101 is positioned as proximal as possible, but sufficiently distant to permit the entrapment of the embolic material and to allow easy handling and positioning of a stent-delivery system, and finally, stent deployment. Furthermore, positioning must be without excessive advancement of the ECD which would allow embolic material to escape into lateral branches 1034, if positioned beyond vessel bifurcations.

Step 3—the ECD 1101 is activated (i.e., the “umbrella” is opened or the “balloon” inflated), after which the cGW is withdrawn, in order to avoid its jailing between the stent and the vessel wall after stent deployment (FIG. 57a).

Step 4—a conventional stent-delivery system is advanced using the ECD 1101 as a guidewire (FIG. 58).

Step 5—the stent-delivery system is inflated and the stent deployed (FIG. 59).

Step 6—debris and vasospastic substances, released during the stenting procedure, and entrapped by the ECD, are removed: with suction using a dedicated probe which has been advanced until it is contiguous with the occlusive “balloon”, or withdrawn within the “umbrella”, after its closure (FIG. 60).

As clearly described, this technique presents some drawbacks:

ECD 1101, used as guidewire, give low support to the delivery systems especially when they are positioned very proximally;

a guidewire 1024 repositioning could be needed after the stent deployment because of procedural complications (such as dissections) or in order to treat other lesions which come to light only after they has been re-opened. This procedure takes time and can be hazardous and unsuccessful.

Therefore, leaving the guidewire 1024 for the 1 duration of the procedure would be preferable.

All of this is easily performed with the proposed device 1 which allows to ride both a cGW 1024 (represented with a broken line in FIGS. 58, 59 and 60) and a ECD 1101 or 1102 simultaneously, utilizing the apical port 1014 and a lateral or side port 1015 provided on a side of the elongated body of the expansion means 1005 (FIGS. 50, 51).

Therefore, we can leave a distally positioned cGW 1024 for the duration of the procedure, as an “auxiliary wire” to give more support to the delivery system and to avoid re-crossing the stented lesion, should this become necessary.

This proposed device also offers a significant clinical advantage in the treatment of a thrombotic occlusion involving the ostium of a branch, or just proximal to a vessel bifurcation (very frequent cases), as shown in the following steps:

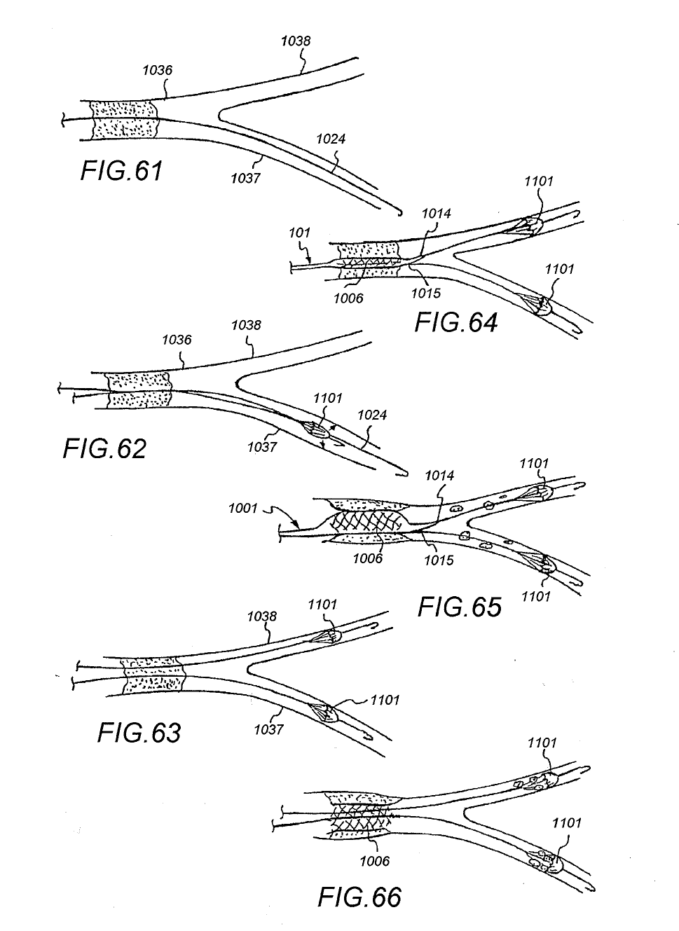

Step 1—the occlusion is crossed using a cGW 1024 as a “trailblazer” (FIG. 61);

Step 2—a first ECD 1101 is advanced into a first branch 1037 (FIG. 62);

Step 3—a second ECD 1101 is advanced into a second branch 1038, and both ECD are activated after the cGW 1024 withdrawal (FIG. 63);

Step 4—the proposed stent-delivery device 1001 is 1 advanced and positioned with the simultaneous use of both ECD's as guidewires (FIG. 64);

Step 5—the stent is deployed and the vessel patency and the blood flow restored (FIG. 65);

Step 6—the debris and any substance released during the procedure, entrapped by the 1002 ECD's, are finally removed (FIG. 66).

Further clinical condition, where the device is extremely useful, is in an ostial lesion at the origin of the right coronary artery or a saphenous graft. In this case the engagement of a guidecatheter 1103 is impossible due to the narrowing of the lumen. Therefore the guidecatheter 1103 is positioned free in the middle of the aortic lumen, opposite the ostium, where there are a continual cardiac-cycle related movements of both the guidecatheter 1103 and the delivery system 1001.

In such circumstances the stent positioning and deployment, using the known devices, is necessarily imprecise and may improperly be implanted, or may be the cause of failure of the procedure. So, often times, these clinical situations are referred to surgeon for aorto-coronary bypass grafting.

Utilizing the proposed device 1001, it is possible to attain a precise positioning and deployment. The proposed method comprises: positioning of a first guidewire 1024 in the diseased vessel suitable to fit it in the apical port 1014 of a proposed device guidewire lumen: then positioning of a second guidewire 1025 free in the aortic lumen and fitting said free guidewire in a proposed device side port 1015^{XIV}, just proximal to a stent 1006 crimped down on the delivery system. In this way, the proposed device 1001 can be advanced in the right coronary artery until the emerging second guidewire 1025 blocks the delivery system with the proximal edge of the stent 1006 perfectly aligned with the aortic wall. By maintaining a constant, even push until the stent delivery system (balloon) is activated (inflated), it is possible to attain a stable positioning within the ostial lesion and, therefore, the proper deployment of a stent.

A further method of employment of the proposed device is in the stenting of bifurcated lesions, where the proposed device 1001 allows the operator to implant simultaneously two stents 1006^{I} and 1006^{II}, perfectly flanked with their proximal edges on the same level, utilizing a known “V” or “kissing” technique.

After having positioned guidewires 1024, 1025 in the respective branches 1037 and 1038, a first guidewire 1024 is fitted in a first device through its apical port 1014.

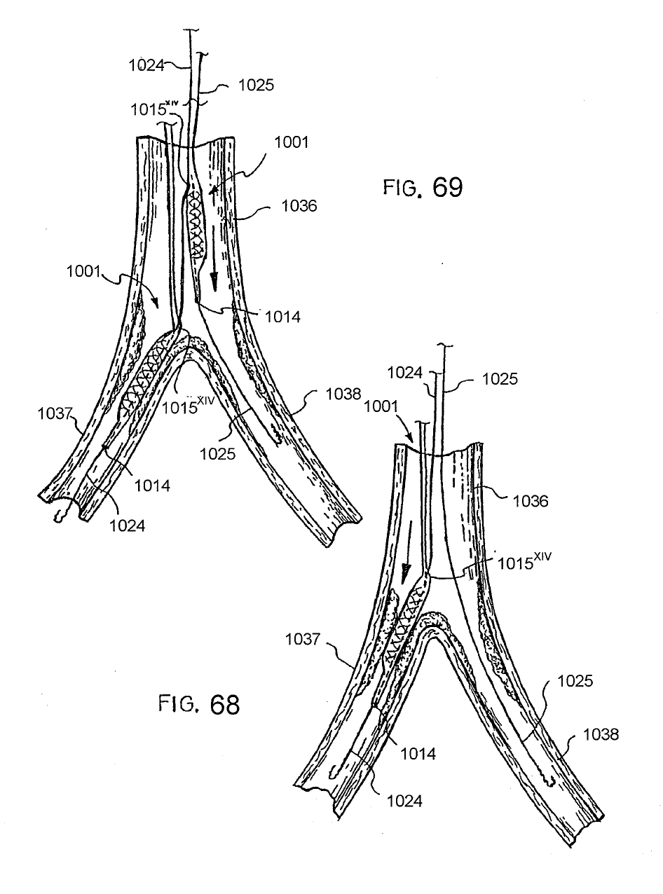

The same guidewire exits the device through a side port 1015^{XIV}, proximal to the stent, and the first device is then advanced in the first branch 1037 (FIG. 68).

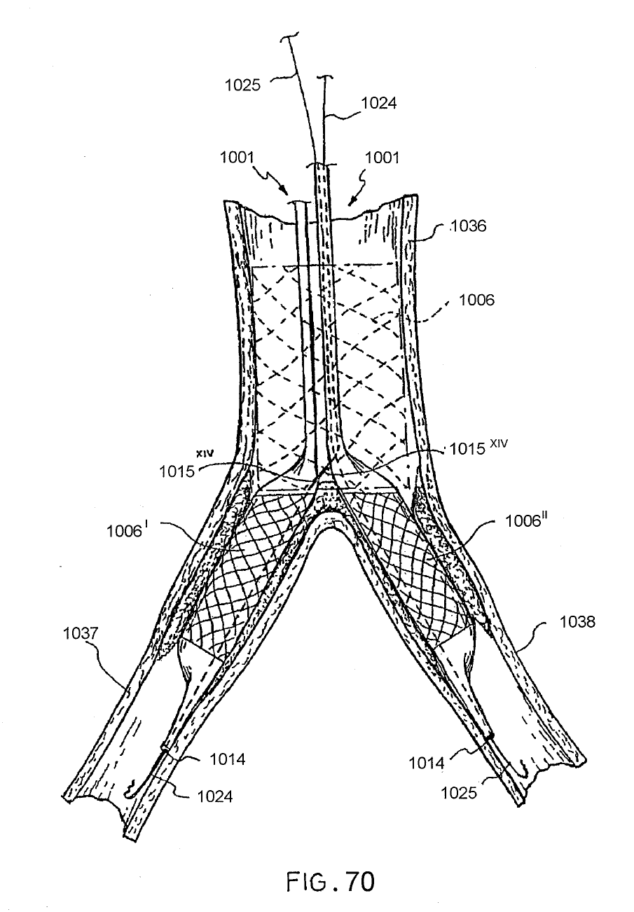

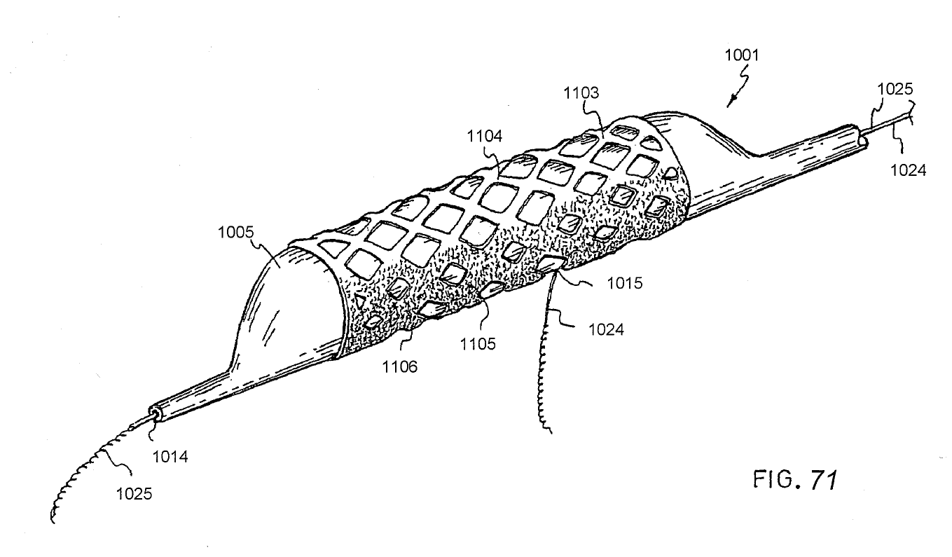

A second guidewire 1025 is received in a second device through its apical port 1014. The first guidewire 1024, received in the first device, is subsequently fitted in the second device through its side port 1015^{XIV} proximal to the stent (FIG. 69).

This second device is then advanced until it is “automatically” blocked when its side port 1015^{XIV} arrives at vessel bifurcation, where the two guidewires 1024, 1025 diverge. With a gentle pulling back of the first device, the respective side ports 1015^{XIV} will be perfectly aligned and held in position by the first guidewire 1024, which exits the first device and re-enters the second device.

In this way, the stents 1006I and 1006II, mounted on two devices will necessarily be positioned with the proximal stent edges at the same level and with a complete coverage of the vascular “carina” between the two branches (FIG. 70).

Contrary to the “V” and “kissing” technique used with traditional balloons, the proposed device allows an “automatic” and precise positioning of paired stents, avoiding approximations, or that one of the two delivery systems is dislodged by the other during inflation of the balloons.

The proposed method of deployment is extremely efficient, particularly if employed subsequently a preliminary deployment of a stent 1006 in the parent vessel, just proximal to the bifurcation: or implanting in the two branches dedicated stents having proximal-angled edges. In this way the coverage of the lesion is improved, avoiding overlapping of the stents, and with a complete coverage of the plaque (FIG. 70).

It is a further advantage that the proposed device has the possibility to rotate in a controlled manner along its longitudinal axis. In this way it is possible to properly orient and deploy stents. Thus, even without a bifurcated lesion, with a guidewire 1024 previously deployed in a side-branch (i.e. in a septal or diagonal branch) it is possible to implant dedicated stents 1103 with variable structures along their circumference (i.e.: struts 1104, 1105 of variable widths or with different drug coatings 1106, and cells, with different diameter or dimensions, provided in different region of the stent) thereby allowing a specific treatment of selected areas in a single lesion.

A person skilled in the art could make numerous changes and adaptations to the preferred embodiment of the endolumenal device described above or substitute elements with others functionally equivalent, in order to meet contingent and specific requirements, without however departing from the scope of the following claims.

What is claimed is:

1. An endolumenal device for delivering and positioning an endolumenal expandable prosthesis for a bifurcation between a principal conduit and at least one secondary conduit, comprising:
an elongated body having a central longitudinal axis, a proximal portion, and a distal portion;
the distal portion of said elongated body comprising an expansion device configured to expand symmetrically relative to the central longitudinal axis and having a longitudinally extended active portion removably engageable with the endolumenal expandable prosthesis and adapted to adjust said prosthesis from a radially collapsed condition to a radially expanded condition;
a guidewire lumen extending at least partially along said elongated body, said guidewire lumen disposed within a wall structure of said expansion device and having a plurality of distal ports extending through the wall structure of the elongate body and adapted to receive through said ports a portion of at least one guidewire positionable with its distal portion in said principal conduit or in said at least one secondary conduit,
wherein said guidewire lumen comprises a distal apical port in an approximately central position relative to said expansion device considered in cross section at right angles to the central longitudinal axis; and
wherein a central region of the expansion device comprises a free volume.

2. The endolumenal device of claim 1, wherein the proximal portion comprises a hypotube and the distal portion comprises an elongate neck portion disposed between the hypotube and the expansion device, the elongate neck comprising a plurality of lumens providing fluid communication between the hypotube and the expansion device and access for a guidewire.

3. The endolumenal device of claim 1, further comprising a joint disposed between the hypotube and the expansion device.

4. The endolumenal device of claim 3, wherein the joint comprises an end-to-end connection between the hypotube and the elongate neck.

5. The endolumenal device of claim 4, wherein the joint comprises an area of overlap between a distal portion of the hypotube and a proximal portion of the elongate neck.

6. The endolumenal device of claim 1, wherein the distal portion of the hypotube is inserted into the proximal portion of the elongate neck to a location distal of a proximal port of the guidewire lumen.

7. The endolumenal device of claim 1, wherein the proximal port of the guidewire lumen has a tapered configuration such that the guidewire port has a distal end and a proximal end and a distal opening of the hypotube is tapered such that the hypotube distal opening has a distal end and a proximal end, wherein at least the distal end of the hypotube distal opening is distal of the distal end of the proximal port of the guidewire lumen.

8. The endolumenal device of claim 1, wherein the elongate neck comprises a proximal portion of the expansion means.

9. The endolumenal device of claim 1, wherein the expansion device comprises a distal tapered portion defined by a wall structure and the guidewire lumen defines a distal portion extending within the wall structure and through the distal tapered portion of the expansion device.

10. The endolumenal device of claim 1, wherein the expansion device comprises a proximal tapered portion defined by a wall structure and the guidewire lumen defines a proximal portion extending within the wall structure and through the proximal tapered portion of the expansion device.

11. The endolumenal device of claim 1, wherein the expansion device comprises a substantially symmetrical unexpanded state.

12. The endolumenal device of claim 1, wherein the expansion device comprises a first pleat and a second pleat in the unexpanded state, the first pleat having a concave portion configured to receive a wall structure through which a guidewire lumen extends, the second pleat configured to receive a convex surface of the first pleat.

13. The endolumenal device of claim 1, further comprising three distal ports extending through the wall structure of the expansion device.

14. The endolumenal device of claim 1, further comprising a second guidewire lumen formed of a separate elongate member, the second elongate member being disposed within an inflation lumen of the expansion device.

15. A method of treating a patient, comprising:
providing a catheter assembly having an elongate body and an expansion device coupled with a distal portion of the elongate body, the expansion device comprising a wall surrounding a cavity and a single guidewire lumen extending from a proximal port to a distal port and also extending at least partially within the wall of the expansion device, the single guidewire lumen having a plurality of side ports disposed along the expansion device sized to provide access for a guidewire to the guidewire lumen;
positioning a guidewire in the vasculature of a patient such that a distal portion thereof is disposed in a portion of a first blood vessel located adjacent to and distal of a bifurcation;
positioning the catheter assembly over the guidewire such that the distal portion of the guidewire extends through one of the side-ports and into the portion of the first blood vessel while at least a portion of the expansion device is located in a portion of a second blood vessel located adjacent to and distal of the bifurcation; and
expanding the expansion device such that the wall is substantially symmetrically disposed around a longitudinal axis extending through the distal port of the guidewire lumen and such that the side ports move radially away from the longitudinal axis.

16. The method of claim 15, further comprising urging a divergence formed between the distal portion of the guidewire and a portion of the expansion device into contact with the bifurcation to confirm the position of at least a portion of the expansion device.

17. The method of claim 15, wherein the catheter is positioned over the guidewire such that the distal portion of the guidewire extends through a side port located adjacent to a proximal end of the expansion device such that a central portion of the expansion device is disposed just distal of the bifurcation.

18. The method of claim 15, wherein the catheter is positioned over the guidewire such that the distal portion of the guidewire extends through a side port located adjacent to a distal end of the expansion device such that a central portion of the expansion device is disposed just distal of the bifurcation.

19. The method of claim 15, further comprising positioning a second guide within the vasculature such that a distal portion thereof extends within the second blood vessel and thereafter, advancing the distal port of the catheter assembly over a proximal portion of the second guidewire until at least a portion of the expansion device is positioned within the second blood vessel.

20. The method of claim 15, wherein the expansion device comprises a proximal tapered portion extending between the proximal most side port and the elongate body and further comprising retracting the catheter assembly over the second guidewire such that the proximal tapered portion dilates plaque within the vasculature.

21. The method of claim 19, further comprising extending the proximal portion of the second guidewire through one of the side ports of the expansion device such that the first and second guidewires do not overlap within the single guidewirelumen.

22. The method of claim 15, wherein a distal portion of the first guidewire extends through the proximal-most side port into the first blood vessel and a proximal portion of the second guidewire extends through the proximal-most side port and through the vasculature.

23. The method of claim 22, wherein a distal portion of the first guidewire extends through a side port disposed centrally on the expansion device into the first blood vessel and a proximal portion of the second guidewire extends through the centrally disposed side-port and through the vasculature.

24. The method of claim 22, wherein a distal portion of the first guidewire extends through the distal-most side port into the first blood vessel and a proximal portion of the second guidewire extends through the distal-most side port and through the vasculature.

25. The method of claim 22, wherein a distal portion of the first guidewire extends through the proximal-most side port into the first blood vessel and a proximal portion of the second guidewire extends through a side-port located distal of the proximal-most side port and through the vasculature.

26. The method of claim 22, wherein a distal portion of the first guidewire extends through a side port disposed centrally on the expansion device into the first blood vessel and a proximal portion of the second guidewire extends through a side-port located distal of the centrally disposed side port and through the vasculature.

27. The method of claim 22, wherein a distal portion of the first guidewire extends through the proximal-most side port into the first blood vessel and a proximal portion of the second guidewire extends through the distal-most side-port and through the vasculature.

28. A method of manufacturing an endolumenal device, comprising:
providing an elongate member having a substantially continuous construction including a first lumen and a second lumen;
forming at least one of the first lumen and second lumens of the elongate members into functional catheter lumens by applying pressure to at least one of the first or second lumens under controlled temperature conditions.

29. The method of claim 28, wherein the controlled temperature conditions includes cold deforming at least one of the first lumen and the second lumen inside a mold to form at least one of the guidewire lumen and the expansion device.

30. The method of claim 28, wherein the providing step include providing an elongate body in which the first lumen is sized and shaped to receive a guidewire and the second lumen is smaller than the first lumen, and the forming step comprises enlarging the second lumen to an expanded size corresponding to the expanded size of an endovascular prosthesis.
